(19) United States
(12) Patent Application Publication
(10) Pub. No.: US 2008/0132886 A1
(43) Pub. Date: Jun. 5, 2008

(54) USE OF FRACTIONAL EMR TECHNOLOGY ON INCISIONS AND INTERNAL TISSUES

(75) Inventors: Richard Cohen, Sherborn, MA (US); Michael H. Smotrich, Andover, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: PALOMAR MEDICAL TECHNOLOGIES, INC., Burlington, MA (US)

(21) Appl. No.: 11/952,745

(22) Filed: Dec. 7, 2007

Related U.S. Application Data
(63) Continuation-in-part of application No. 11/097,841, filed on Apr. 1, 2005.
Application No. 11/097,841 is a continuation-in-part of application No. 11/098,000, filed on Apr. 1, 2005.
Application No. 11/098,000 is a continuation-in-part of application No. 11/098,036, filed on Apr. 1, 2005.
Application No. 11/098,036 is a continuation-in-part of application No. 11/098,015, filed on Apr. 1, 2005.
(60) Provisional application No. 60/868,982, filed on Dec. 7, 2006.
(60) Provisional application No. 60/561,052, filed on Apr. 9, 2004.
(60) Provisional application No. 60/614,382, filed on Sep. 29, 2004.
(60) Provisional application No. 60/641,616, filed on Jan. 5, 2005.
(60) Provisional application No. 60/620,734, filed on Oct. 21, 2004.
(60) Provisional application No. 60/561,052, filed on Apr. 9, 2004.
(60) Provisional application No. 60/614,382, filed on Sep. 29, 2004.
(60) Provisional application No. 60/641,616, filed on Jan. 5, 2005.
(60) Provisional application No. 60/620,734, filed on Oct. 21, 2004.
(60) Provisional application No. 60/561,052, filed on Apr. 9, 2004.
(60) Provisional application No. 60/614,382, filed on Sep. 29, 2004.
(60) Provisional application No. 60/641,616, filed on Jan. 5, 2005.
(60) Provisional application No. 60/620,734, filed on Oct. 21, 2004.
(60) Provisional application No. 60/561,052, filed on Apr. 9, 2004.
(60) Provisional application No. 60/614,382, filed on Sep. 29, 2004.
(60) Provisional application No. 60/641,616, filed on Jan. 5, 2005.
(60) Provisional application No. 60/620,734, filed on Oct. 21, 2004.

(51) Int. Cl. A61B 18/18

(52) U.S. Cl. 606/34

(57) ABSTRACT

Methods of treatment of tissue with electromagnetic radiation (EMR) to produce lattices of EMR-treated islets in the tissue are disclosed. Specifically, methods of treating internal hard and soft tissues, such as but not limited to organs, bones, muscles, tendons, ligaments, vessels and nerves, with such EMR-treated islets are described. Also disclosed are devices and systems for producing lattices of EMR-treated islets in tissue, and cosmetic and medical applications of such devices and systems.

### RELATED APPLICATIONS

### Claim of Priority

This application claims the benefit of U.S. Provisional Application No. 60/868,982, filed Dec. 7, 2006, which is incorporated herein by reference.

This application is a continuation-in-part application of U.S. application Ser. Nos. 11/097,841, 11/098,000, 11/098,036, and 11/098,015, each of which was filed Apr. 1, 2005 and entitled “Methods and products for producing lattices of EMR-treated islets in tissues, and uses therefore” and each of which claims priority to U.S. Provisional Application No. 60/561,052, filed Apr. 9, 2004, U.S. Provisional Application No. 60/614,382, filed Sep. 29, 2004, U.S. Provisional Application No. 60/641,616, filed Jan. 5, 2005, and U.S. Provisional Application No. 60/620,734, filed Oct. 21, 2004. Each of these applications and provisional applications are incorporated herein by reference in their entirety.

### BACKGROUND OF THE INVENTION

1. Field of the Invention

The devices and methods disclosed herein relate to the treatment of soft and hard tissues with electromagnetic radiation (EMR) to produce lattices of EMR-treated islets in the tissue to stimulate and facilitate repair and healing in a controlled fashion. The devices and methods also relate to systems for producing such lattices of EMR-treated islets in tissue, and cosmetic, medical and other applications of such devices, methods and systems.

2. Description of the Related Art

Electromagnetic radiation, particularly in the form of laser light, has been used in a variety of cosmetic and medical applications, including uses in dermatology, dentistry, opthalmology, gynecology, otorhinolaryngology and internal medicine. For most dermatological applications, the EMR treatment can be performed with a device that delivers the EMR to the surface of the targeted tissues. For applications in internal medicine, the EMR treatment is typically performed with a device that works in combination with an endoscope or catheter to deliver the EMR to internal surfaces and tissues. As a general matter, the EMR treatment is typically designed to (a) deliver one or more particular wavelengths (or a particular continuous range of wavelengths) of EMR to a tissue to induce a particular chemical reaction, (b) deliver EMR energy to a tissue to cause an increase in temperature, (c) deliver EMR energy to a tissue to damage or destroy cellular or extracellular structures, or (d) deliver EMR energy to a tissue to activate an exogenous substance that has been injected (as in the case of some cancer treatments) or topically applied (as in the case of some acne treatments).

EMR treatments of various tissues, including internal tissues and tissues involved in surgical, medical, therapeutic, post-operative, and other procedures, have some of the same limitations as similar cosmetic treatments that apply EMR to the surface of skin to perform, e.g., resurfacing or other procedures. For example, the wavelengths typically utilized for selective photothermolysis may be highly scattered and/or highly absorbed, which limits the ability to selectively target body components and, in particular, limits the depths at which treatments can be effectively and efficiently performed. Much of the energy applied to a target region may be either scattered such that it does not reach the body component undergoing treatment, or may be absorbed by overlying or surrounding tissue. Thus, larger and more powerful EMR sources may be required in order to achieve a desired therapeutic result. However, increasing power may cause undesired and potentially dangerous heating of tissue.

In some cases when treating internal tissues, such as nerves or small structures involved in certain surgical procedures, bulk heating of the entire tissue may be detrimental. Similarly, certain tissues may have already been damaged by trauma or during the course of surgical or other procedures, and may thus be more susceptible than healthy tissue to unwanted damage from the application of too much EMR. Additionally, internal tissues may benefit from the application of EMR using techniques that promote the repair of tissue, accelerate the healing process, and/or accentuate the healing process. The use of new devices and techniques may promote healing and/or prevent damage to such tissue.

In the cosmetic field for the treatment of various skin conditions, methods and devices have been developed that irradiate or cause damage in a portion of the tissue area and/or volume being treated. These methods and devices have become known as fractional technology. Fractional technology is thought to be a safer method of treatment of skin for cosmetic purposes, because the damage occurs within smaller sub-volumes or islets within the larger volume being treated. The tissue surrounding the islets is spared from the damage. Because the resulting islets are surrounded by neighboring healthy tissue the healing process is thorough and fast.

### SUMMARY OF THE INVENTION

One aspect of the invention is a method of treating internal tissue that includes accessing an internal tissue volume to be treated, and irradiating portions of the internal tissue volume with electromagnetic radiation. The electromagnetic radiation causes the heated portions to form islets of treated tissue surrounded by untreated tissue.

Preferred embodiments of this aspect of the invention may include one or more of the following. The internal tissue is accessed by one of an incision, an open wound, and an orifice of a body cavity. The internal tissue is a tissue from the group muscle, cartilage, ligaments, bone, fat, dermis, blood vessels, nervous tissue, gastrointestinal, heart, lungs, kidney, gall bladder, and liver. The heated portions may be ablated, coagulated, and/or denatured. The heated portions may alternatively be heated without further damage to the tissue in the heated portions. The treated tissue may be welded. The treated tissue may be a surgical incision and/or be composed of two portions of tissue joined during surgery. The heated portions may be heated substantially simultaneously or may be scanned. The treated portions may be irradiated for a time that is greater than the thermal relaxation time of the tissue volume to be treated.

Another aspect of the invention is a method of treating internal tissue that includes inserting a treatment device into the internal tissue to be treated; causing the treatment device to transmit electromagnetic radiation from the device to portions of the internal tissue; and forming subvolumes of damaged tissue corresponding to the irradiated portions of the internal tissue, wherein the subvolumes are separated by undamaged tissue.

Preferred embodiments of this aspect of the invention may include one or more of the following. The treatment device may include a cannula or a catheter. The internal tissue to be treated is a blood vessel, and the treatment device is inserted into a lumen of the blood vessel. The ratio of the subvolumes of treated tissue to the volume of internal tissue being treated is between about 0.1% and about 90%, or more specifically may be about 10% to about 50%, or even more specifically may be about 10% to about 30%.

Another aspect of the invention is a method of performing a treatment on a volume located at area and depth coordinates of an internal tissue of a patient, which includes providing a source of treatment radiation; and applying treatment radiation from the source to an optical system providing multiple foci for concentrating said radiation to at least one depth within said depth coordinate and to selected areas within said area coordinates of said volume such that following application of the treatment radiation three dimensionally located treatment portions are formed at the foci in said volume separated from one another by untreated portions of said volume.

Another aspect of the invention is a method for performing a treatment on a volume located at area and depth coordinates of an internal tissue by irradiating portions of the volume including providing a source of treatment radiation; precooling the internal tissue over at least part of the area coordinate to a selected temperature for a selected duration, the selected temperature and duration being sufficient to cool the internal tissue to a depth below the depth coordinate to a temperature below normal body temperature of the internal tissue; and applying the treatment radiation to an optical system having a plurality of foci which concentrates said radiation to at least one depth coordinate and to selected areas within said area coordinate to define treatment portions at said foci in said volume following application of the treatment radiation, said treatment portions being less than said volume, each said treatment portion being within untreated portions and being substantially surrounded by cooled internal tissue separating said treatment portion from other treatment portions.

Another aspect of the invention is a device for performing a treatment on a volume of internal tissue located at area and depth coordinates of a patient's skin. The device may include a source of treatment radiation, an optical system to which treatment radiation from said source is applied. The optical system may provide a plurality of foci for concentrating said treatment radiation to at least one depth in said volume of internal tissue and to selected areas of said volume, with the at least one depth and the areas defining three dimensional treatment portions at the foci in the volume within untreated portions of the volume. The device further may include a controller for selectively activating the source so as to successively irradiate the plurality of foci.

Preferred embodiments of this aspect of the invention may include one or more of the following. The device may include a cooling system configured to cool the volume of internal tissue. The cooling system may be configured to cool the volume of internal tissue during operation to a selected temperature and to a selected depth. The device may include cannula or a catheter each configured to emit radiation from a portion thereof.

In various embodiments, the methods and devices described herein provide for the fractional treatment of various hard and soft tissues such as internal tissues, including without limitation, muscle (including smooth, cardiac and striated muscle), cartilage, ligaments, bone, blood vessels, nervous tissue, tissue of the gastrointestinal system (including the esophagus, stomach, small intestine, large intestine and colon) and tissue of various organs such as the heart, lungs, kidney, gall bladder, and liver. Such tissues may be treated, for example, during a surgical or medical procedure through an incision or using a catheter or other devices. Such tissues can also be treated using non-surgical and non-medical procedures, for example, as during therapy or the treatment of post-operative and other wounds.

### BRIEF DESCRIPTION OF THE DRAWINGS

The following drawings are illustrative of embodiments and are not meant to limit the scope of the invention as encompassed by the claims.

FIGS. 1A-1C are semi-schematic perspective and side views respectively of a section of muscle tissue and of equipment positioned thereon for practicing one embodiment.

FIG. 2 is a schematic diagram of a device for treating internal tissue.

FIG. 3 is a schematic diagram of an alternate embodiment of a device for treating internal tissue.

FIG. 4 is a side schematic view of some components that can be used in some aspects.

FIGS. 5 and 5A are schematic views other embodiments of the invention in which an endoscope is used to create EMR-treated islets in the walls of a blood vessel.

FIGS. 6A and 6B are top views of various matrix arrays of cylindrical lenses, some of which are suitable for providing a line focus for a plurality of target portions.

FIGS. 7A-7C are top views of various matrix arrays of cylindrical lenses, some of which are suitable for providing a line focus for a plurality of target portions.

FIGS. 8A-8D are cross-sectional or side views of one layer of a matrix cylindrical lens system suitable for delivering radiation in parallel to a plurality of target portions.

FIG. 9A is a side view of yet another embodiment.

FIGS. 9B to 9E are enlarged, side views of the distal end of the embodiment of FIG. 9A.

FIG. 10A is a side view of yet another embodiment.

FIGS. 10B and 10C are enlarged, side views of the distal end of the embodiment of FIG. 10A.

FIG. 11 is a side view of yet another embodiment.

FIG. 12A is a side view of an embodiment using a diode laser bar.

FIG. 12B is a perspective view of a diode laser bar that can be used in the embodiment of FIG. 12A.

FIG. 12C is a side view of yet another embodiment, which uses multiple diode laser bars.

FIG. 12D is a side view of yet another embodiment, which uses multiple diode laser bars.

FIG. 12E is a side view of yet another embodiment, which uses multiple optical fibers to couple optical energy.

FIG. 13A is a side view of another embodiment.

FIG. 13B is a perspective view of a light source and optical fiber that can be used along with the embodiment of FIG. 13A.

FIG. 13C is a side view of an embodiment using a fiber bundle.

FIG. 13D is a bottom view of the embodiment of FIG. 13C.

FIG. 13E is an enlarged, side view of a distal end of one of the embodiments of 13A-13D.

FIG. 14A is a side view of another embodiment, which uses a fiber bundle.

FIG. 14B is a side view of another embodiment, which uses a phase mask.

FIG. 14C is a side view of another embodiment, which uses multiple laser rods.

FIG. 15 is a bottom view of another embodiment, which uses one or more capacitive imaging arrays.

FIG. 16 is a side view of another embodiment, which uses a motor capable of moving a diode laser bar within a hand piece.

FIG. 17 is a top view of one embodiment of a diode laser bar.

FIG. 18 is a side cross-sectional view of the diode laser bar of FIG. 17.

FIGS. 19A-19C are top views of three optical systems involving arrays of optical elements suitable for use in delivering radiation in parallel to a plurality of target portions.

FIGS. 20A-21D are side views of various lens arrays suitable for delivering radiation in parallel to a plurality of target portions.

FIGS. 22A-22D are side views of Fresnel lens arrays suitable for delivering radiation in parallel to a plurality of target portions.

FIGS. 23A-23C are side views of holographic lens arrays suitable for use in delivering radiation in parallel to a plurality of target portions.

FIGS. 24A-24B are side views of gradient lens arrays suitable for use in delivering radiation in parallel to a plurality of target portions.

FIGS. 25A-25C are a perspective view and cross-sectional side views, respectively, of a two layer cylindrical lens array suitable for delivering radiation in parallel to a plurality of target portions.

FIGS. 26-29 are side views of various optical objective arrays suitable for use in concentrating radiation to one or more target portions.

FIGS. 30A-35 are side views of various deflector systems suitable for use with the arrays to move to successive target portions.

FIGS. 36 and 37 are side views of two different variable focus optical systems.

FIG. 38 is a perspective view of another embodiment for creating treatment islets.

FIG. 39 is a perspective view of another embodiment.

FIG. 40 is a perspective view of yet another embodiment.

### DETAILED DESCRIPTION

When using electromagnetic radiation (EMR) to treat tissues, there are many advantages to producing lattices of EMR-treated islets in the tissue rather than large, continuous regions of EMR-treated tissue. The lattices are periodic patterns of islets in one, two or three dimensions in which the islets correspond to local maxima of EMR-treatment of tissue. The islets are separated from each other by non-treated tissue (or differently- or less-treated tissue). The EMR-treatment results in a lattice of EMR-treated islets which have been exposed to a particular wavelength or spectrum of EMR, and which is referred to herein as a lattice of “optical islets.” When the absorption of EMR energy results in significant temperature elevation in the EMR-treated islets, the lattice is referred to herein as a lattice of “thermal islets.” When an amount of energy is absorbed that is sufficient to significantly disrupt cellular or intercellular structures, the lattice is referred to herein as a lattice of “damage islets.” When an amount of energy (usually at a particular wavelength) sufficient to initiate a certain photochemical reaction is delivered, the lattice is referred to herein as a lattice of “photochemical islets.” By producing EMR-treated islets rather than continuous and/or uniform regions of EMR-treatment, more EMR energy can be delivered to an islet without producing a thermal islet or damage islet, and/or the risk of bulk tissue damage can be lowered.

EMR-treated islets can also be formed within an area or volume of treated tissue, for example, where the entire tissue area and/or volume is treated with a relatively lower intensity of EMR having a same or different wavelength while the islets are formed by treating portions of the area and/or volume using EMR having a higher intensity. One skilled in the art will recognize that many combinations of parameters are possible that will result in such local maxima of EMR-treatment within the tissue.

When using electromagnetic radiation (EMR) to treat tissues, whether for purposes of photodynamic therapy, photobiomodulation, photobiostimulation, photobiosuspension, thermal stimulation, thermal coagulation, thermal ablation or other applications, there are substantial advantages to producing lattices of EMR-treated islets in the tissue rather than large, continuous regions of EMR-treated tissue. The EMR-treated tissues can be any hard or soft tissues for which such treatment is useful and appropriate, including but not limited to dermal tissues, mucosal tissues (e.g., oral mucosa, gastrointestinal mucosa), ophthalmic tissues (e.g., retinal tissues), neuronal tissue, vaginal tissue, glandular tissues (e.g., prostate tissue), internal organs, bones, teeth, muscle tissue, blood vessels, tendons and ligaments.

The lattices are periodic patterns of islets in one, two or three dimensions in which the islets correspond to local maxima of EMR-treatment of tissue. The islets are separated from each other by non-treated tissue (or differently- or less-treated tissue). The EMR-treatment results in a lattice of EMR-treated islets which have been exposed to a particular wavelength or spectrum of EMR, and which is referred to herein as a lattice of “optical islets.” When the absorption of EMR energy results in significant temperature elevation in the EMR-treated islets, the lattice is referred to herein as a lattice of “thermal islets.” When an amount of energy is absorbed that is sufficient to significantly disrupt cellular or intercellular structures, the lattice is referred to herein as a lattice of “damage islets.” When an amount of energy (usually at a particular wavelength) sufficient to initiate a certain photochemical reaction is delivered, the lattice is referred to herein as a lattice of “photochemical islets.”

By producing EMR-treated islets rather than continuous regions of EMR-treatment, untreated regions (or differently- or less-treated regions) surrounding the islets can act as thermal energy sinks, reducing the elevation of temperature within the EMR-treated islets and/or allowing more EMR energy to be delivered to an islet without producing a thermal islet or damage islet and/or lowering the risk of bulk tissue damage. Moreover, with respect to damage islets, it should be noted that the regenerative and repair responses of the body occur at wound margins (i.e., the boundary surfaces between damaged and intact areas) and, therefore, healing of damaged tissues is more effective with smaller damage islets, for which the ratio of the wound margin to volume is greater.

As described more fully below, the percentage of tissue volume which is EMR-treated versus untreated (or differently- or less-treated) can determine whether optical islets become thermal islets, damage islets or photochemical islets. This percentage is referred to as the “fill factor”, and can be decreased by increasing the center-to-center distance(s) of islets of fixed volume(s), and/or decreasing the volume(s) of islets of fixed center-to-center distance(s).

Because untreated tissue volumes act as a thermal sink, these volumes can absorb energy from treated volumes without themselves becoming thermal or damage islets. Thus, a relatively low fill factor can allow for the delivery of high fluence energy to some volumes while preventing the development of bulk tissue damage. Finally, because the untreated tissue volumes act as a thermal sink, as the fill factor decreases, the likelihood of optical islets reaching critical temperatures to produce thermal islets or damage islets also decreases (even if the EMR power density and total exposure remain constant for the islet areas).

The embodiments described below provide improved devices and systems for producing lattices of EMR-treated islets in tissues, and improved cosmetic and medical applications of such devices and systems in plastic surgery, physical medicine, orthopedic medicine, neurology, neurosurgery, dermatology, dentistry, opthalmology, gynecology, otorhinolaryngology and internal medicine, for example, during a surgery in which an open incision exposes the tissue to be treated or in combination with endoscope and catheter procedures. Although the devices, systems and methods are described in detail for internal medical applications, they can be used for treatment of any tissue surface or subsurface areas to which EMR can be delivered.

### Categories of EMR-Treated Islets

The embodiments described herein relate to the creation of a multiplicity of treated volumes of the tissue which are separated by untreated volumes. The multiplicity of volumes can be described as defining a “lattice,” and the treated volumes, because they are separated by untreated volumes, can be described as “islets” within the tissue. Depending upon the nature of the treatment, in particular the amount of energy transfer to the islets, the degree of heating of the tissue, or the wavelength(s) of the energy, four different categories of lattices can be produced: lattices of optical islets (LOI), lattices of thermal islets (LTI), lattices of damage islets (LDI), and lattices of photochemical islets (LPCI). These different categories of EMR-treated islets, devices and systems for producing such EMR-treated islets, and cosmetic and medical applications for such devices and systems are separately discussed in detail below. As used herein, the terms “treatment islet,” “islets of treatment,” and “EMR-treated islets” are used interchangeably to mean any of the categories of islets described below.

A. Optical Islets

EMR-treatment of completely or partially isolated volumes or islets of tissue produces a lattice of EMR-treated islets surrounded by untreated volumes. Although the islets can be treated with any form of EMR, they are referred to herein as “optical” islets for convenience, as many embodiments include the use of EMR within the ultraviolet, visible and infra-red spectrum. Other forms of EMR may be useful, including, without limitation, microwave, radio frequency, low frequency and EMR induced by direct current.

As noted above, when the total energy transfer per unit cross-sectional area (i.e., fluence) or the rate of energy transfer per unit cross-sectional area (i.e., flux) becomes sufficiently high, the tissue of an optical islet will be heated, resulting in a thermal islet. If the temperature increase is sufficiently high, the tissue of a thermal islet will be damaged, resulting in a damage islet. Thus, although all thermal islets and damage islets are also optical islets, not all optical islets are thermal islets or damage islets. In some embodiments, as described below, it can be desirable to produce optical islets without producing thermal or damage islets. In such embodiments, the fill factor can be decreased in order to provide a greater volume of untreated tissue to act as a thermal sink.

B. Thermal Islets

EMR-treatment of isolated volumes or islets of tissue can produce a lattice of thermal islets with temperatures elevated relative to those of surrounding untreated volumes. Thermal islets result when energy is absorbed by an EMR-treated optical islet significantly faster than it is dissipated and, therefore, significant heating occurs.

Heating can result from the absorbance of EMR by water present throughout a volume of treated tissue, by endogenous chromophores present in selected cells or tissue(s) (e.g., melanin, hemoglobin), by exogenous chromophores pre-administered or applied within the tissue (e.g., tattoo ink, ALA) or, as described below, by exogenous chromophores applied to the tissue.

A lattice of thermal islets is a time-dependent phenomenon. If absorptive heating occurs at too great a rate or for too long a period, heat will begin to diffuse away from the EMR-treated islets and into the surrounding untreated tissue volumes. As this occurs, the thermal islets will spread into the untreated volumes and, ultimately, the thermal islets will merge and result in bulk heating. By using a sufficiently short pulse width relative to the temperature relaxation time of the target, it is possible to avoid merging or overlapping of thermal islets in a lattice.

C. Damage Islets

EMR-treatment of isolated volumes or islets of tissue can produce a lattice of damage islets surrounded by volumes of undamaged tissue (or differently- or less-damaged tissue). Damage islets result when the temperature increase of an EMR-treated thermal islet is sufficient to result in protein coagulation, thermal injury, photodisruption, photoablation, or water vaporization. Depending upon the intended use, damage islets with lesser degrees of damage (e.g., protein coagulation, thermal injury) or greater degrees of damage (e.g., photodisruption, photoablation, or water vaporization) may be appropriate. As before, damage can result from the absorbance of EMR by water present throughout a volume of treated tissue, by endogenous chromophores present in selected cells or tissue(s) tissue (e.g., melanin, hemoglobin), by exogenous chromophores within the tissue (e.g., tattoo ink, ALA) or, as described below, by exogenous chromophores applied to the surface of the tissue.

In some embodiments, the damage islets are thermal injuries with coagulation of structural proteins. Such damage can result when, for example, the light pulse duration varies from several microseconds to about 1 sec, but the peak tissue temperature remains below the vaporization threshold of water in the tissue (Pearce et al. (1995), in Optical-Thermal Response of Laser-Irradiated Tissue, Welch et al., eds. (Plenum Press, New York), pp. 561-606). The degree of damage is a function of the tissue temperature and the duration of the thermal pulse, and can be quantified by any of several damage functions known in the art. In the description below, for example, the damage function yielding the Arrhenius damage integral (Pearce et al. (1995), in Optical-Thermal Response of Laser-Irradiated Tissue, Welch et al., eds. (Plenum Press, New York), pp. 561-606; Henriques (1947), Arch. Pathol. 43:480-502) is employed. Other mechanisms and models of damage islet formation can apply to embodiments with relatively short and intense pulses, particularly in connection with photodisruption, photoablation, and water vaporization.

D. Photochemical Islets

EMR-treatment of isolated volumes or islets of tissue can produce a lattice of photochemical islets surrounded by volumes of tissue in which a photochemical reaction has not been induced. The photochemical reaction can involve endogenous biomolecules or exogenous molecules. For example, exposure of the tissue to certain wavelengths of EMR can result in increased melanin production and “tanning” through the activation of endogenous biomolecules and biological pathways. Alternatively, for example, exogenous molecules can be administered in photodynamic therapy, and activation of these molecules by certain wavelengths of EMR can cause a systemic or localized therapeutic effect.

### Treatment Parameters.

In practice, a variety of different treatment parameters relating to the applied EMR can be controlled and varied according to the particular cosmetic or medical application. These parameters include, without limitation, the following:

A. The Shape of EMR-Treated Islets.

The optical islets can be formed in any shape which can be produced by the devices described below, limited only by the ability to control EMR beams within the tissue. Thus, depending upon the wavelength(s), temporal characteristics (e.g., continuous versus pulsed delivery), and fluence of the EMR; the geometry, incidence and focusing of the EMR beam; and the index of refraction, absorption coefficient, scattering coefficient, anisotropy factor (the mean cosine of the scattering angle), and the configuration of the tissue layers; and the presence or absence of exogenous chromophores and other substances, the islets can be variously-shaped volumes extending from the surface of the tissue through one or more layers, or extending from beneath the surface of the tissue through one or more layers, or within a single layer. If the beams are not convergent, such beams will define volumes of substantially constant cross-sectional areas in the plane orthogonal to the beam axis (e.g., cylinders, rectanguloids). Alternatively, the beams can be convergent, defining volumes of decreasing cross-sectional area in the plane orthogonal to the central axis of the beams (e.g., cones, pyramids). The cross-sectional areas can be regular in shape (e.g., ellipses, polygons) or can be arbitrary in shape. In addition, depending upon the wavelength(s) and fluence of an EMR beam, and the absorption and scattering characteristics of a tissue for the wavelength(s), an EMR beam may penetrate to certain depths before being initially or completely absorbed or dissipated and, therefore, an EMR-treated islet may not extend through the entire depth of the tissue but, rather, may extend between the surface and a particular depth, or between two depths below the surface.

Generally, though not necessarily, the lattice is a periodic structure of islets, and can be arranged in one, two, or three dimensions. For instance, a two-dimensional (2D) lattice is periodic in two dimensions and translation invariant or non-periodic in the third. For example, and without limitation, there can be layer, square, hexagonal or rectangle lattices. The lattice dimensionality can be different from that of an individual islet. A single row of equally spaced cylinders is an example of a 1D lattice of 3D islets. For certain applications, an “inverted” lattice can be employed, in which islets of intact tissue are separated by areas of EMR-treated tissue and the treatment area is a continuous cluster of treated tissue with non treated islands.

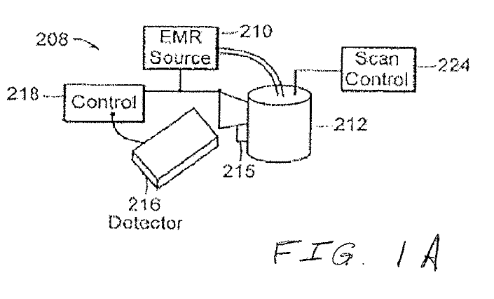

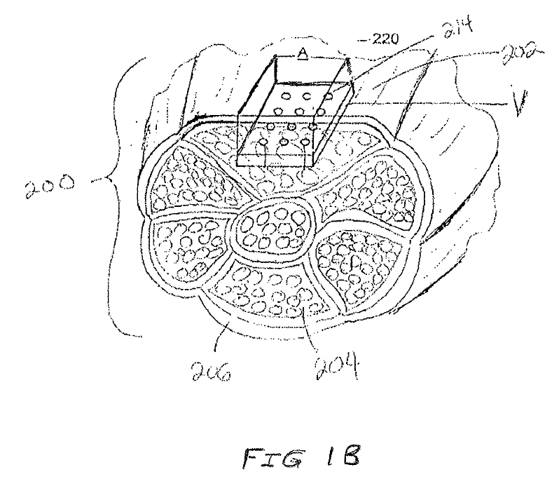

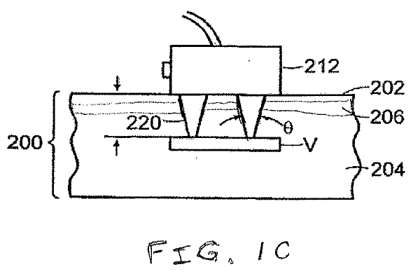

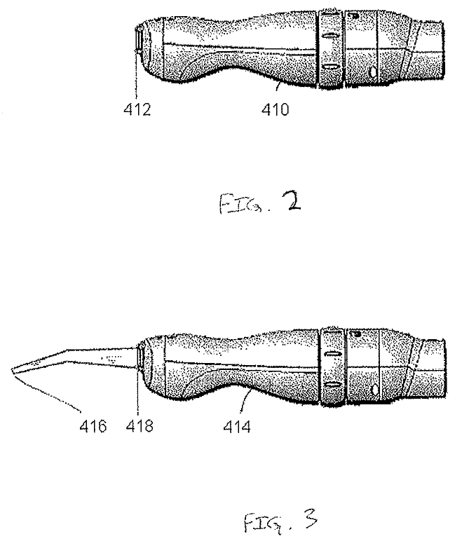

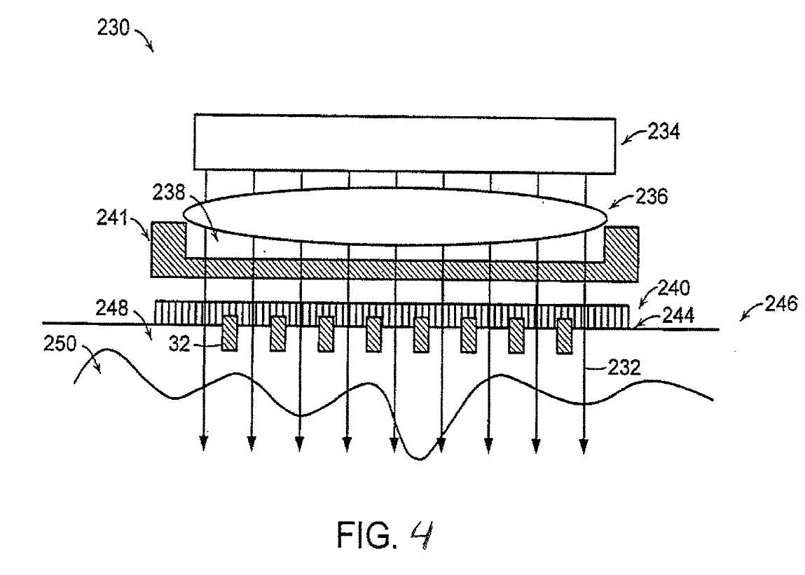

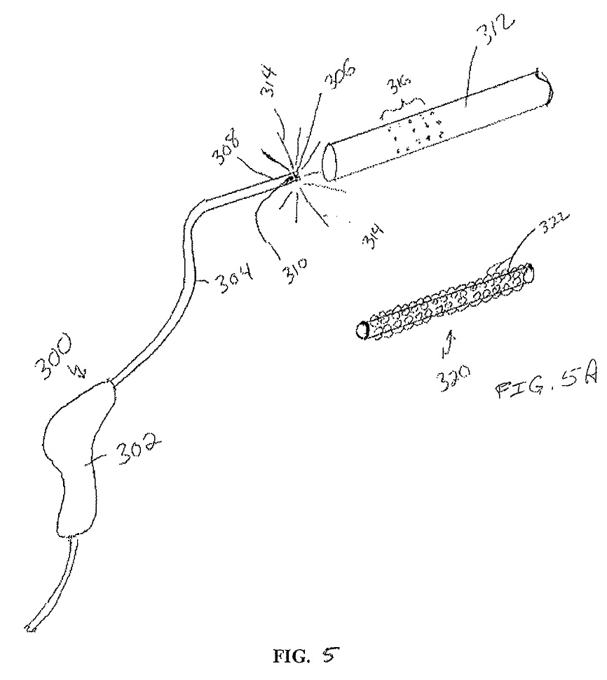

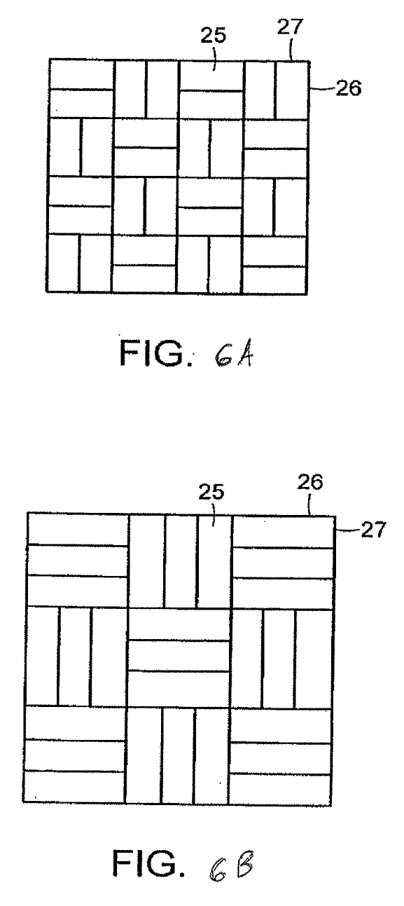

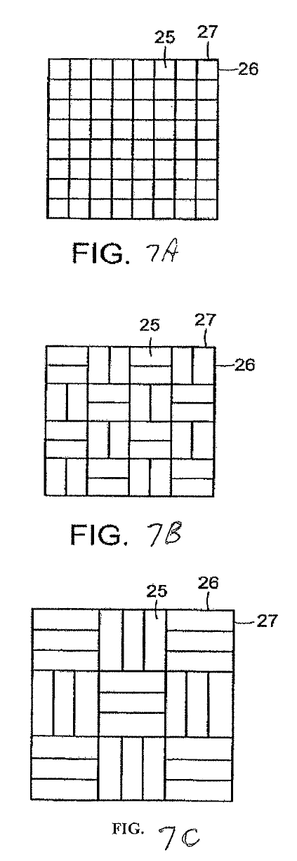

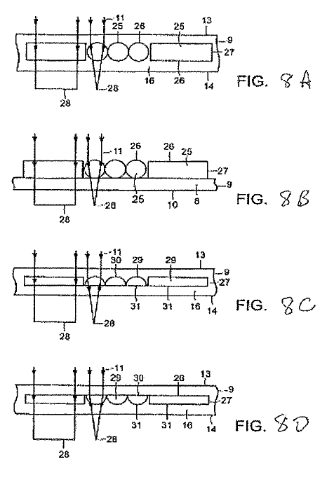

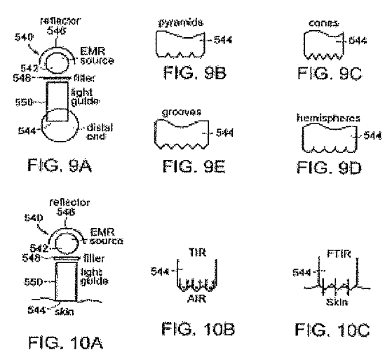

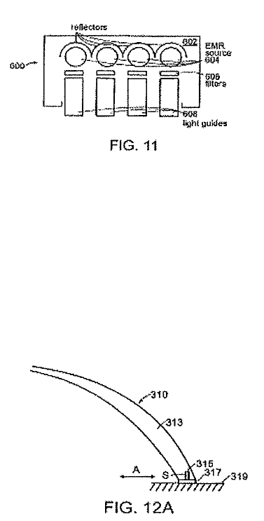

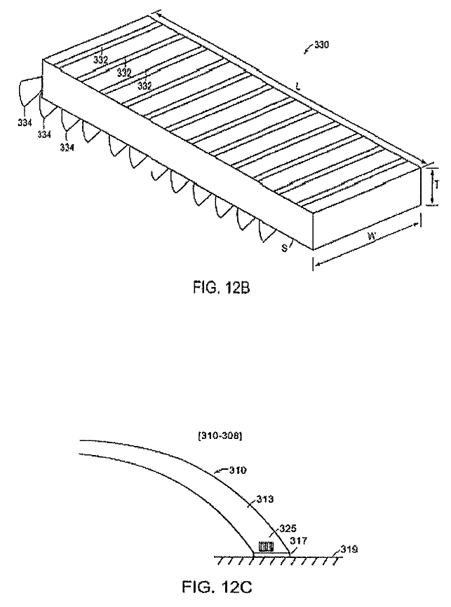

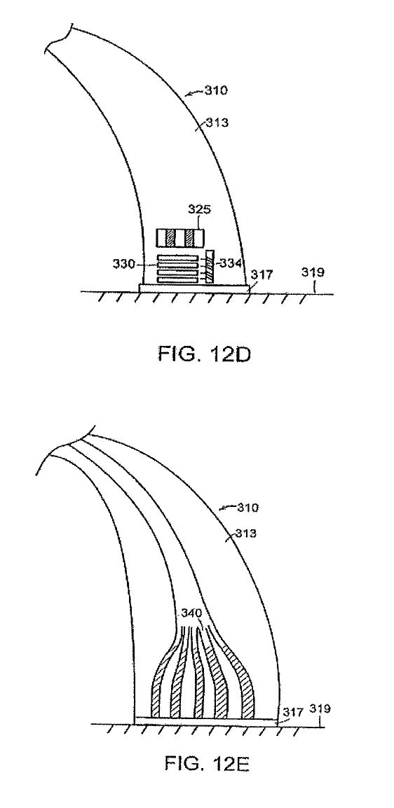

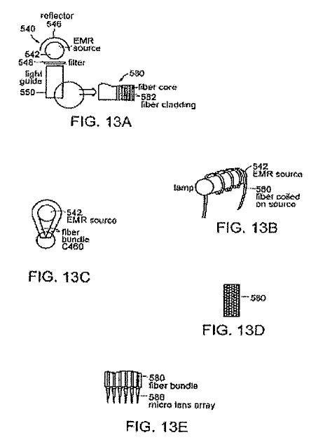

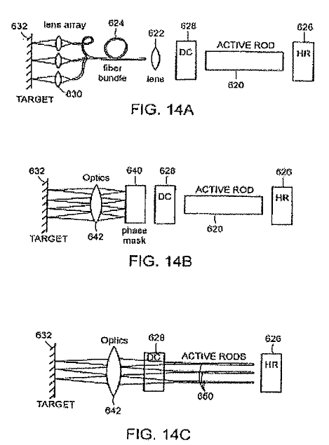

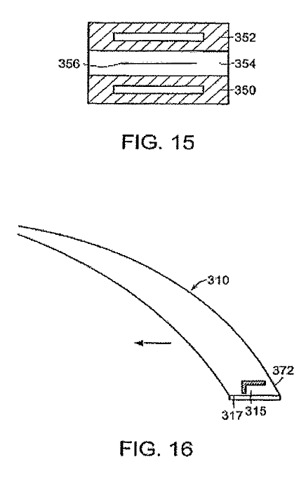

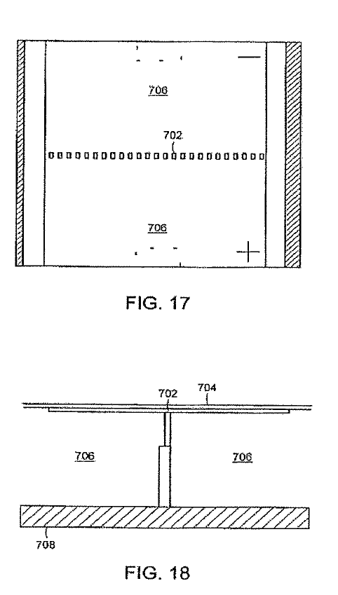

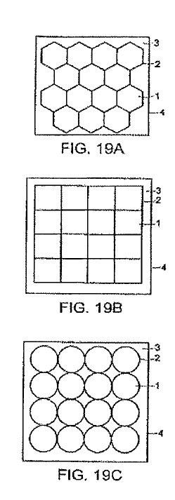

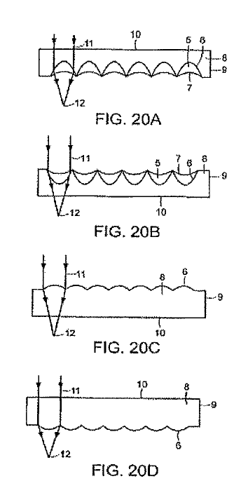

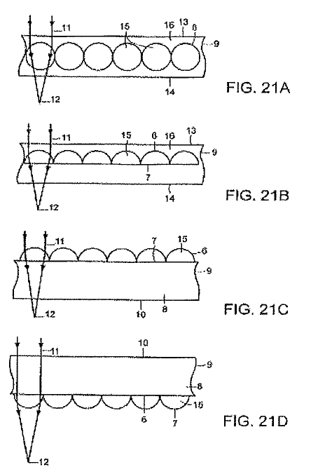

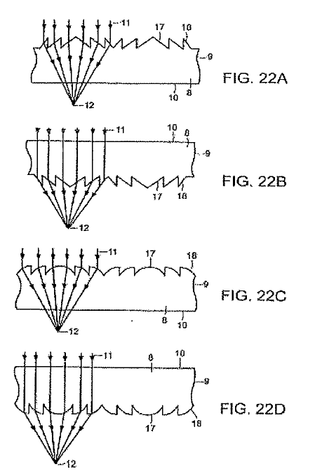

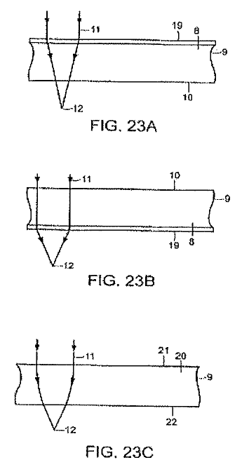

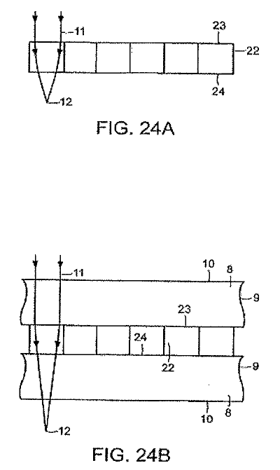

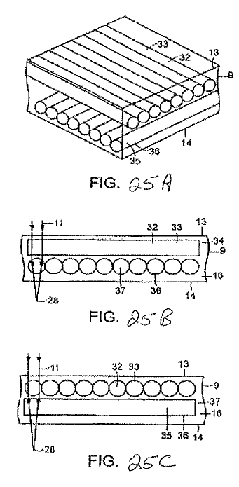

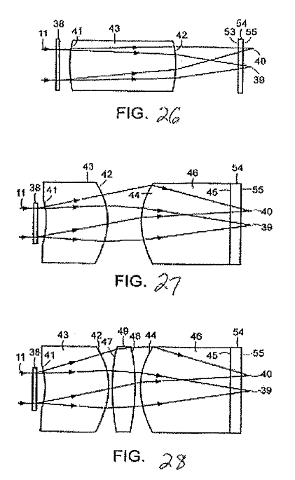

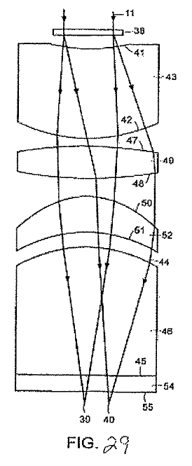

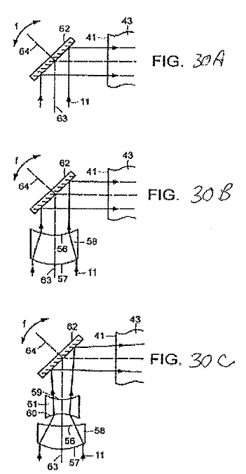

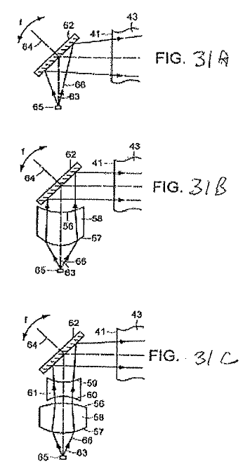

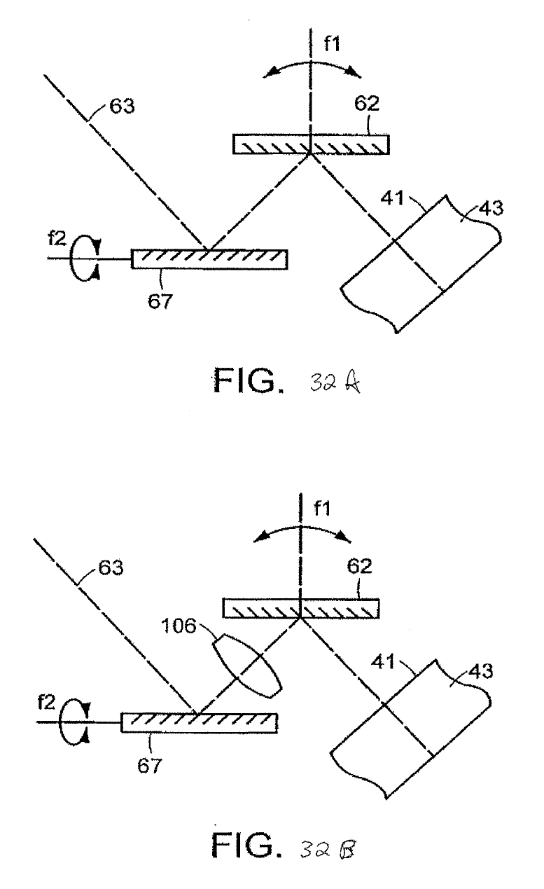

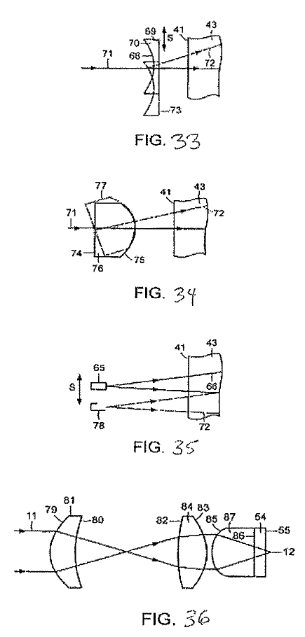

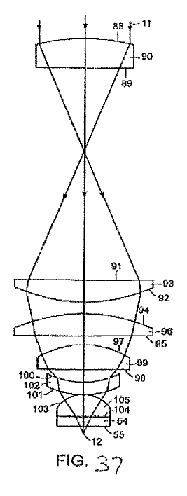

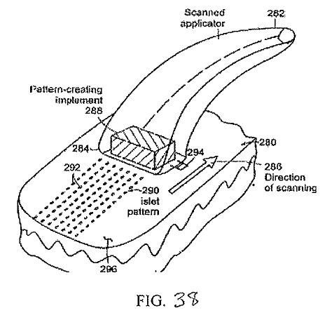

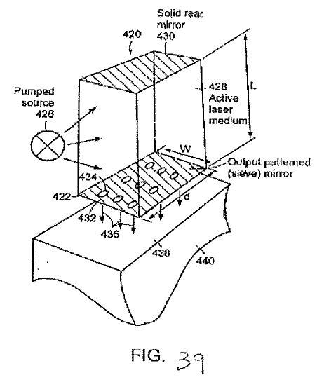

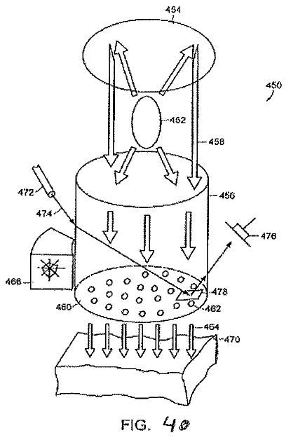

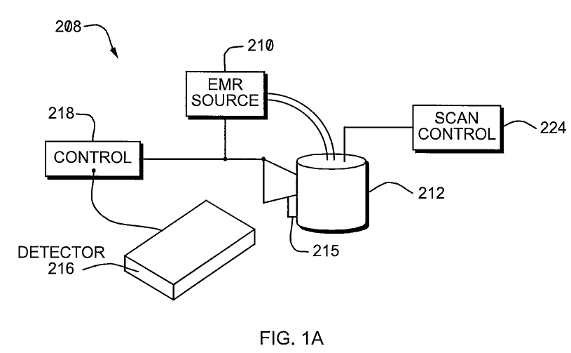

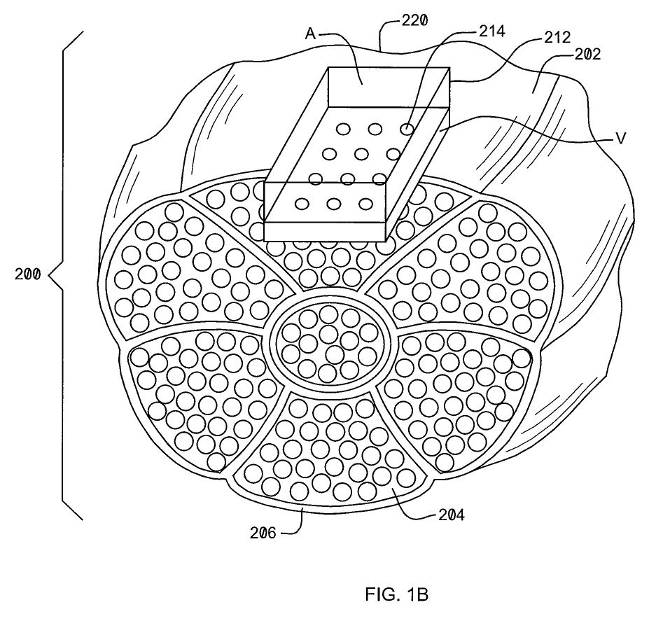

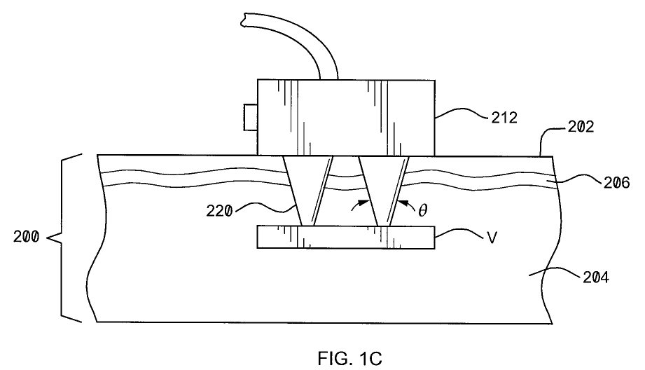

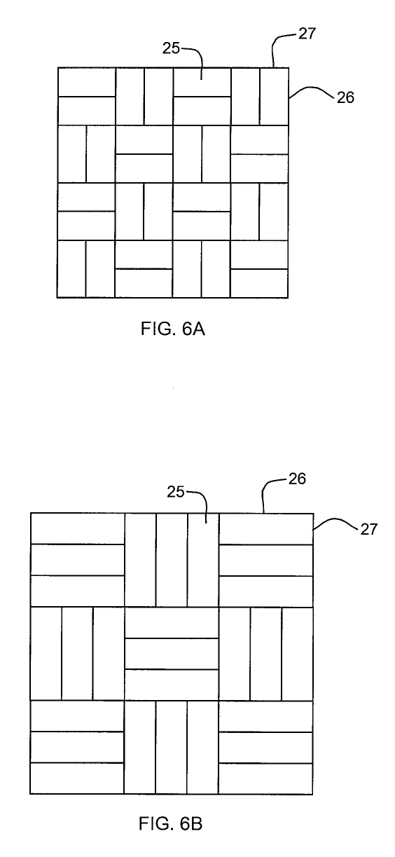

Referring to FIGS. 1A-1C, each of the treated volumes can be a relatively thin disk, as shown, a relatively elongated cylinder (e.g., extending from a first depth to a second depth), or a substantially linear volume having a length which substantially exceeds its width and depth, and which is oriented substantially parallel to the tissue surface. The orientation of the lines for the islets 214 in a given application need not all be the same, and some of the lines may, for example, be at right angles to other lines (see for example FIGS. 6A and 6B). Lines also can be oriented around a treatment target for greater efficacy. For example, the lines can be perpendicular to a vessel or parallel to a wrinkle. As shown in FIGS. 1A-1B, islets 214 are subsurface cylindrical volumes. However, many other configurations are possible, such as spheres, ellipsoids, cubes or rectanguloids of selected thickness and starting at or below the surface of the tissue being treated. The islets can also be substantially linear or planar volumes. The shapes of the islets are determined by the combined optical parameters of the beam, including beam size, amplitude and phase distribution, the duration of application and, to a lesser extent, the wavelength.

The parameters for obtaining a particular islet shape can be determined empirically with only routine experimentation. For example, a 1720 nm laser operating with a low conversion beam at approximately 0.005-2 J and a pulse width of 0.5-2 ms, can produce a generally cylindrically shaped islet. Alternatively, a 1200 nm laser operating with a highly converting beam at approximately 0.5-10 J and a pulse width of 0.5-3 sec, can produce a generally ellipsoid-shaped islet.

By suitable control of wavelength, focusing, incident beam size at the surface and other parameters, the islets, regardless of shape, can extend through a volume, can be formed in a single thin layer of a volume, or can be staggered such that adjacent islets are in different thin layers of volume. Most configurations of a lattice of islets can be formed either serially or simultaneously. Lattices with islets in multiple thin layers in a volume can be easily formed serially, for example using a scanner or using multiple energy sources having different wavelengths. Islets in the same or varying depths can be created, and when viewed top-down from the tissue surface, the islets at varying depths can be either spatially separated or overlapping.

The geometry of the islets affects the thermal damage in the treatment region. Since a sphere provides the greatest gradient, and is thus the most spatially confined, it provides the most localized biological damage, and can therefore be preferred for applications where this is desirable. Other geometries that increase the surface to volume ratio of the islets may be preferred for other applications.

B. The Size of EMR-Treated Islets.

The size of the individual islets within the lattices of EMR-treated islets, can vary widely depending upon the intended cosmetic or medical application. As discussed more fully below, in some embodiments it is desirable to cause substantial tissue damage to destroy a structure or region of tissue (e.g., vessel, tendon, or facia) whereas in other embodiments it is desirable to cause little or no damage while administering an effective amount of EMR at a specified wavelength (e.g., photodynamic therapy). As noted above with respect to damage islets, however, the healing of damaged tissues is more effective with smaller damage islets, for which the ratio of the wound margin to volume is greater.

As a general matter, the size of the EMR-treated islets can range from 1 μm to maximum length of targeted tissue in any particular dimension. For example, and without limitation, a lattice of substantially linear islets can consist of parallel islets having a length of approximately 300 mm and a width of approximately 10 μm to 3 mm to treat the length of a blood vessel. As another example, and without limitation, for substantially cylindrical islets in which the axis of the cylinder is orthogonal to the tissue surface, the depth can be approximately 10 μm to 4 mm and the diameter can be approximately 10 μm to 1 mm. For substantially spherical or ellipsoidal islets, the diameter or major axis can be, for example, and without limitation, approximately 10 μm to 1 mm. Thus, in some embodiments, the islets can be used to treat a specific portion of the target tissue surrounding a region of injury or in other embodiments treat the entire target tissue so as to induce a generalized tissue response throughout the target.

When considering the size of the optical, thermal, damage or photochemical islets, it is important to note that the boundaries of the islets may not be clearly demarcated but, rather, may vary continuously or blend into the untreated tissue (or differently- or less-treated tissue). For example, EMR beams are subject to scattering in various tissues and, therefore, even beams of coherent light will become diffuse as they penetrate through multiple layers of cells or tissues. As a result, optical and photochemical islets typically may not have clear boundaries between treated and untreated volumes. For some parameters, the transition from treated to untreated tissue will be quick and the boundaries of the islet will be well defined. For other parameters, the transition will be more gradual and less well defined. Similarly, thermal islets typically will exhibit a temperature gradient from the center of the islet to its boundaries, and untreated tissue surrounding the islet also will exhibit a temperature gradient due to conduction of heat. Finally, damage islets can have irregular or indistinct boundaries due to partially damaged cells or structures or partially coagulated proteins. As used herein, therefore, the size of an islet within a lattice of islets, refers to the size of the islet as defined by the intended minimum or threshold amount of EMR energy delivered. This amount is expressed as the minimum fluence, Fₘᵢₙ, and is determined by the nature of the cosmetic or medical application. For example, for photodynamic therapy, Fₘᵢₙ can be determined by the minimum fluence necessary to cause the desired photochemical reaction. Similarly, for increasing the permeability of the tissue, Fₘᵢₙ can be determined by the minimum fluence necessary to achieve the desired tissue temperature, and for destroying tissue, Fₘᵢₙ can be determined by the minimum fluence necessary to ablate the tissue or vaporize water. In each case, the size of the EMR-treated islet is defined by the size of the tissue volume receiving the desired minimum fluence.

Because of the scattering effects of tissue, the minimum size of an EMR-treated islet increases with the targeted depth in the tissue. For a depth of approximately 1 mm into a subject's tissue, the practical minimum diameter or width of a non-ablative islet is estimated to be approximately 100 μm, although much larger islets (e.g., 1-10 mm) are possible. (However, islets smaller than 100 μm are theoretically possible, especially in the context of ablation where scattering effects may be reduced, and such islets are not outside the scope of the embodiments and claims.)

The size of a damage islet can be either smaller or larger than the size of the corresponding optical islet, but is generally larger as greater amounts of EMR energy are applied to the optical islet due to heat diffusion. For a minimum size islet at any particular depth in the tissue, the wavelength, beam size, convergence, energy and pulse width have to be optimized.

C. The Depth of EMR-Treated Islets.

The EMR-treated islets can be located at varying points within a tissue, including surface and subsurface locations, locations at relatively limited depths, and locations spanning substantial depths. The desired depth of the islets depends upon the intended cosmetic or medical application, including the location of the targeted molecules, cells, tissues or intercellular structures.

For example, optical islets can be induced at varying depths in a tissue or organ, depending upon the depth of penetration of the EMR energy, which depends in part upon the wavelength(s) and beam size. Thus, the islets can be shallow islets that penetrate only surface layers of a tissue (e.g., 0-50 μm), deeper islets that span several layers of a tissue (e.g., 50-500 μm), or very deep, subsurface islets ((e.g., 500 μm-5 mm or more). Using optical energy, depths of up to 25 mm can be achieved. Using microwave and radio frequency EMR, depths of several centimeters can be achieved.

For thermal islets or damage islets, subsurface islets can be produced by targeting chromophores present only at the desired depth(s), or by cooling upper layers of a tissue while delivering EMR. For creating deep thermal or damage islets, long pulse widths coupled with surface cooling can be particularly effective.

D. Fill Factor of EMR-Treated Lattices

In a given lattice of EMR-treated islets, the percentage of tissue volume which is EMR-treated is referred to as the “fill factor” or f, and can affect whether optical islets become thermal islets, damage islets or photochemical islets. The fill factor is defined by the volume of the islets with respect to a reference volume that contains all of the islets. The fill factor may be uniform for a periodic lattice of uniformly sized EMR-treated islets, or it may vary over the treatment area. Non-uniform fill factors can be created in situations including, but not limited to, the creation of thermal islets using topical application of EMR-absorbing particles in a lotion or suspension (see below). For such situations, an average fill factor (f_{avg}) can be calculated by dividing the volume of all EMR-treated islets Vᵢ^{islet} by the volume of all tissue Vᵢ^{tissue} in the treatment area,

$${f}_{avg} = {\sum }_{i} \frac{{V}_{i}^{islet}}{{V}_{i}^{tissue}} .$$

Generally, the fill factor can be decreased by increasing the center-to-center distance(s) of islets of fixed volume(s), and/or decreasing the volume(s) of islets of fixed center-to-center distance(s). Thus, the calculation of the fill factor will depend on volume of an EMR-treated islet as well as on the spacing between the islets. In a periodic lattice, where the centers of the nearest islets are separated by a distance d, the fill factor will depend on the ratio of the size of the islet to the spacing between the nearest islets d. For example, in a lattice of parallel cylindrical islets, the fill factor will be:

$$f = {π ( \frac{r}{d} )}^{2} ,$$

where d is the shortest distance between the centers of the nearest islets and r is the radius of a cylindrical EMR-treated islet. In a lattice of spherical islets, the fill factor will be the ratio of the volume of the spherical islet to the volume of the cube defined by the neighboring centers of the islets:

$$f = \frac{4 π}{3} {( \frac{r}{d} )}^{3} ,$$

where d is the shortest distance between the centers of the nearest islets and r is the radius of a spherical EMR-treated islet. Similar formulas can be obtained to calculate fill factors of lattices of islets of different shapes, such as lines, disks, ellipsoids, rectanguloids, or other shapes. (In the art, the fill factor is sometimes determined two dimensionally for convenience, e.g., based on the percentage of the area of EMR-islets formed at the surface of a tissue to the total surface area.)

Because untreated tissue volumes act as a thermal sink, these volumes can absorb energy from treated volumes without themselves becoming thermal or damage islets. Thus, a relatively low fill factor can allow for the delivery of high fluence energy to some volumes while preventing the development of bulk tissue damage. Finally, because the untreated tissue volumes act as a thermal sink, as the fill factor decreases, the likelihood of optical islets reaching critical temperatures to produce thermal islets or damage islets also decreases (even if the EMR power density and total exposure remain constant for the islet areas).

The center-to-center spacing of islets is determined by a number of factors, including the size of the islets and the treatment being performed. Generally, it is desired that the spacing between adjacent islets be sufficient to protect the tissues and facilitate the healing of any damage thereto, while still permitting the desired therapeutic effect to be achieved. In general, the fill factor can vary in the range of 0.1-90%, with ranges of 0.1-1%, 1-10%, 10-30% and 30-50% for different applications. The interaction between the fill factor and the thermal relaxation time of a lattice of EMR-treated islets is discussed in detail below. In the case of lattices of thermal islets, it can be important that the fill factor be sufficiently low to prevent excessive heating and damage to islets, whereas with damage islets it can be important that the fill factor be sufficiently low to ensure that there is undamaged tissue around each of the damage islets sufficient to prevent bulk tissue damage and to permit the damaged volumes to heal.

### Applications of EMR-Treated Islets

EMR-treated islets can be used in a variety of applications in a variety of different organs and tissues. For example, EMR treatments can be applied to tissues including, but not limited to, tissue mucosal tissues (e.g., oral mucosa, gastrointestinal mucosa), ophthalmic tissues (e.g., retinal tissues), tissues of the ear, vaginal tissue, glandular tissues (e.g., prostate tissue), internal organs, muscle tissues, blood vessels, tendons and ligaments. As a general matter, the methods can be used to treat conditions including, but not limited to, lesions (e.g., sores, ulcers), undesired blood vessels, hyperplastic growths (e.g., tumors, polyps, benign prostatic hyperplasia), hypertrophic growths (e.g., benign prostatic hypertrophy), neovascularization (e.g., tumor-associated angiogenesis), arterial or venous malformations (e.g., hemangiomas, nevus flammeus), and undesired pigmentation (e.g., pigmented birthmarks, tattoos). The time for recovery or healing of such damage islets can be controlled by changing the size of the damage islets and the fill factor of the lattice.

For example, the embodiments described herein are particularly suited to treating internal tissues of the body, for example, in surgical and medical applications. As an example, forming EMR-islets during a surgical procedure in which a ligament or tendon is being repaired, by irradiating a portion of the ligament or tendon with light to form a set of islets on the portion of the ligament or tendon that has been irradiated. The treatment will promote faster healing of the ligament, tendon or other tissue. Further, because the ligament or tendon is already being accessed for purposes of the surgical repair, for example, through an incision or using an endoscope, the EMR therapy can be conducted directly on the ligament or tendon (or other internal tissue) without requiring an additional invasive action or procedure such as making an incision solely for the purposes of the EMR therapy. (Of course, while this advantage is desirable for many embodiments, one skilled in the art will readily appreciate that the advantage is not necessary to all embodiments, and that embodiments within the scope of the claims may include invasive aspects, for example, making an incision, solely for the purpose of accessing and treating internal tissues, such as ligaments, bones, tendons, muscles, organs, blood vessels, bones, nerves, etc.) with EMR for the purposes of forming lattices of damage islets.) Although many other applications are possible, several specific applications are discussed below as exemplary embodiments.

A. Surgical and Other Applications Pertaining to Internal Tissues within a Body:

One particularly useful embodiment EMR-treated islets in surgical and other internal applications are small selective microzones of coagulated tissue, which, for example, may have widths of approximately 100 μm, depths of approximately 400 μm and a center-to-center spacing of approximately 500 μm (although many other dimensions are possible). Selective microzones of coagulated tissue can be used for many purposes, for example, to stimulate repair of ligaments, vessels, tendons, etc. as part of surgical or post-surgical treatments to aid in the repair and reconstruction of damaged tissues. The application of microzones of thermal injury to the reattachment zone of a grafted ligament, or to fracture zone of bone, or to a vessel stimulates responses of the hard and soft tissue to heal and repair more quickly. In other cases in which multiple surgeries are required to treat conditions these are often due to incomplete and inadequate healing following initial treatments. Application of fractional thermolysis, in the form of lattices of EMR treatment islets, to the treated tissue stimulates further healing without the complications of more invasive surgical procedures. This may have significant advantage by reducing the impact and need for further surgical procedures and reduce post-surgical complications. Retreatment of the injured tissue using arthroscopic methods can be used in the course of a series of treatments as part of the overall physical medicine therapy leading to a faster, more complete recovery with fewer complications.

Such methods and apparatus are provided for performing a therapeutic treatment on a patient's tissue by concentrating applied radiation of at least one selected wavelength at a plurality of selected, three-dimensionally located, treatment portions, which treatment portions are within non-treatment portions. The ratio of treatment portions to the total volume may vary from 0.1% to 90%, but is preferably less than 50%. Various techniques, including wavelength, may be utilized to control the depth to which radiation is concentrated and suitable optical systems may be provided to concentrate applied radiation in parallel or in series for selected combinations of one or more treatment portions.

When the density and distribution of these is sufficient within the targeted region a generalized recruitment of healing throughout the region appears to be elicited. This has the advantage that repair is elicited without initial loss of function.

Application of EMR to form lattices of islets of tissue injury has the advantage of extending and recruiting the healing needed to more fully and completely restore function to the entire affected tissue. The EMR lattices preferably will be of sufficient density, depth and volume to stimulate cellular reactions throughout the adjacent and affected surrounding tissue, although treatments of less than the entire affected tissue or of lower potency are possible according to certain embodiments. Treatment of the surrounding affected target tissue as well as the affected tissue damaged in the process of access to the target tissue speeds recovery and function. In some embodiments, the EMR treatment islets may be microscopic in size. Additionally, in some applications, the EMR treatment islets may be formed at temperatures below those that produce coagulation or destruction. In still other cases, EMR treatment islets may be formed by ablating or desiccating tissue. In such cases, the EMR treatment islets will still promote healing, believed to be associated with the mechanisms of photobiostimulation and photobiomodulation. (However, regardless of the actual healing mechanism, the application of such EMR treatment islets at such temperatures promote healing.)

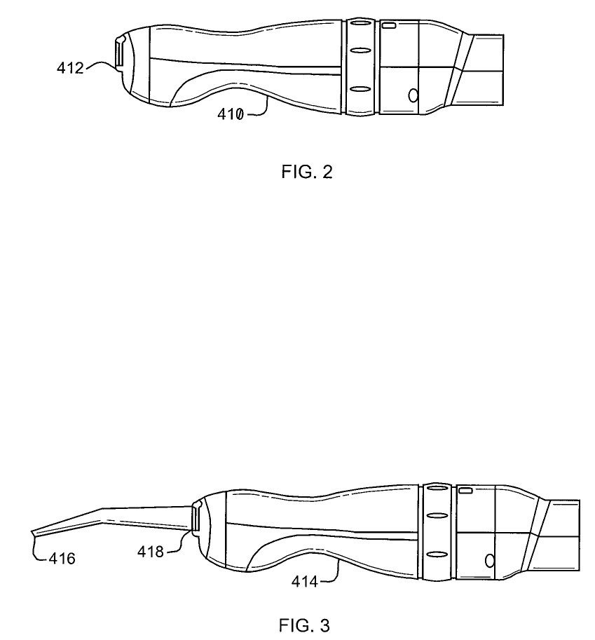

Embodiments described herein are capable performing a therapeutic treatment on internal tissue by concentrating applied radiation of at least one selected wavelength at a plurality of selected, three-dimensionally located, treatment portions, which treatment portions are within non-treatment portions. For example, referring to FIG. 2, a device 410 similar to Palomar Medical Technologies, Inc.'s Lux1540 handpiece may be used. Such a device would have an EMR transmission area 412 of approximately 10 mm. The device 10 emits EMR having a wavelength of 1540 nm with 100 EMR beams per cm², at a fluence of up to 100 mJ per EMR beam, a pulse width of 5-30 ms and a repetition rate of up to 2.5 pulses per second. Many other specifications are possible and other embodiments will have specifications optimized for a particular application.

As an example of another embodiment, referring to FIG. 3, device 414 has a much smaller area 416 for transmitting EMR located at the end of an extended, and also has a curved neck 418 is shown. The electromagnetic radiation is transmitted along curved neck 418 by a waveguide contained within neck 418. Additional optical elements may be required in device 414 to produce a homogenous output at area 416 due to the curved neck 418. Thus, while the curved neck 418 may improve the ergonomics of device 414, a device having a straight neck may be preferable to simply the optical system. The device 414 otherwise operates in a manner similar to the device 410.

In operation, device 410, or a device of similar size, may be used to treat relatively larger areas of tissue, such as repaired muscle and tendons, or a relatively large section of bone. Device 414, or a device of similar size, may be used to perform treatments in a more precise fashion, for example, treating damaged vessels, or nerves of a wound zone in a spinal cord.

Extremely small EMR transmission areas may be used for even finer applications, and may be small enough to produce only several small microscopic or nearly microscopic EMR-treatment islets. For example, in one embodiment, a device having an optical fiber coupled to a micro-lens array may be used. The micro-lens array may be manufactured as discussed herein, but using nano or nano-like technologies to create a lens array of very small size. Using such small and even microscopic or nearly microscopic devices to create EMR-islets, fine and delicate treatments can be performed on very small tissue volumes and structures. For, for example, such devices may be used for treating and/or stimulating a nerve bundle or an individual nerve cell or group of nerve cells or increasing the permeability of a membrane or thin sheath or other similar tissue structure or treating or performing a procedure on small structures in the body such as auditory bones of the ear or valves and other structures in the heart. Many other embodiments are possible.

The specific wavelength, focal depth and intensity will be based upon the intended use. The differences in tissue properties, size, thickness and constituent properties will need to be considered. Size and shape of the handpiece may also be designed to reach and limit properly the zone of tissue to which the EMR is applied.

B. Improving Healing of Incisions, Points of Reattachment, and Other Wounds:

The creation of lattices of damage islets can be used to decrease the time needed for the healing of wounds by recruitment of tissue surrounding the wound margin to participate more fully in the repair and healing process with minimal to no negative impact on the structure and function of the surrounding tissue's capability to perform its normal function. In surgical procedures, tissue is often removed and dissected prior to reattachment and repair. Also in order to gain access to the intended surgical site other structure needs to be opened and cut. These tissues may also need to be reattached.

Using convention surgical techniques and procedures, the recovery site and wound zone is limited to the tissue at the opposing sides of the incision. Much of the ability to restore function, however, depends upon surrounding tissue response and repair. Examples include cases in which a section of vein is removed for grafting to another body site or reattachment of ligaments following a significant tear. The ability to recruit repair mechanisms in adjacent tissue and all along the affected length of the vessel, connective tissue or organ is limited by the vast distances from the incision and attachment site. The natural chemical signals that mediate the natural healing and repair responses of the tissue leading to upregulation and regrowth are at best weak if at all present due to the extensive distance involved to reach the adjacent tissue. In some cases, scar tissue may form and has potential to inhibit and limit repair and restoration of tissue strength and function.

Application of EMR to form lattices of treatment islets, however, promotes recovery by extending and recruiting the healing needed to more fully and completely restore function to throughout the length, breadth and extent of the entire affected tissue.

As one example, in a case where a blood vessel is damaged and a section of the blood vessel is remove prior to rejoining the ends of the blood vessel, EMR-treated islets can be created to promote the overall healing of all or a substantial portion of the affected length of the blood vessel. Following the repair, the vessel may be stressed due to the trauma as well as from the fact that the vessel was stretched to allow the vessel to be repaired. In such a case, EMR-treated islets can be used to speed the vessel's recovery even along portions that are remote from the site of the injury. To provide the treatment, a catheter or similar device can be inserted into the vessel and drawn along the affected length of the vessel while emitting EMR to create the islets or the vessel could be treated along an extensive proximal and distal length at the time of resection.

C. Treating Subcutaneous Tissue Scars:

The creation of lattices of damage islets can be used to treat hypertrophic scars by inducing shrinkage and tightening of the scar tissue, and replacement of abnormal connective tissue with normal connective tissue. Tissue may be treated according to different regimes to alleviate, reduce and/or prevent scarring. For example, an area of tissue, such as skin or a vessel, requiring a surgical incision or procedure can be treated prior to the surgery, in some cases just prior to the surgery and in other cases well in advance of the surgery such as several weeks prior. Such prior treatments will stimulate a healing response in the tissue where the incision is to be formed, which will improve post-surgical healing of the incision and reduce the amount of scarring.

Tissue may also be treated contemporaneously at the time of surgery, for example, while an incision is open. Similarly, a tendon, muscle, blood vessel or other tissue can be treated at a location where the tendon, muscle, blood vessel or other tissue is joined or otherwise repaired to reduce or eliminate the amount of scarring at the site of the repair.

Similarly, scar tissue may be treated after it is formed in subsequent procedures or during rehabilitation or therapy to reduce or eliminate the scar tissue or prevent the further formation of scar tissue.

D. Ablation or Welding of Internal Tissue

The creation of lattices of damage islets can be used in order to damage or destroy or induce healing responses of internal tissue to treat various conditions. The methods and devices can also be used to weld tissues together by creating islets to form the welded areas in the tissue surrounded by healthy tissues. The methods and devices can also be used to ablate a surface of the tissue. (The surface of the tissue can be the naturally occurring surface, and can also be a surface that is created, for example, by cutting or otherwise altering the tissue during a treatment or procedure.

### Products and Methods for Producing Lattices of EMR-Treated Islets

A. Exemplary Embodiments

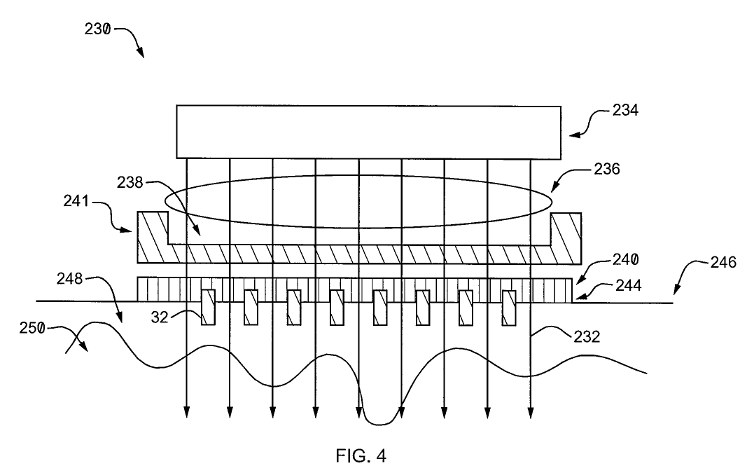

FIG. 4 shows a broad overview schematic of an apparatus 100 that can be used in one embodiment to produce islets of treatment in the tissue. For this apparatus 230, optical energy 232 from a suitable energy source 234 passes through optical device 236, filter 238, cooling mechanisms 240, 242, and cooling or heating plate 244, before reaching tissue 246 (i.e., the subject's tissue). Each of these components is described in greater detail below. Generally, however, the EMR from the energy source 234 is focused by the optical device 236 and shaped by masks, optics, or other elements in order to create islets of treatment. In some embodiments, certain of these components, such as, for example, filter 328 where a monochromatic energy source is utilized or optics 236, may not necessarily be present. In other embodiments, the apparatus may not contact the tissue. In yet another embodiment, there is no cooling mechanism such that there is only passive cooling between the contact plate and the tissue.

A suitable optical impedance matching lotion or other suitable substance may be applied between plate 244 and tissue 246 to provide enhanced optical and thermal coupling, although this may not be required. Furthermore, many internal tissue will have sufficient moisture to provide optical coupling with the device, and the parameters of the device may be optimized to provide impedance matching in those cases, if required or desired. For surgical procedures, any such lotion or substance must be suitable for use within a body. Tissue 246, as shown in FIG. 4, is divided into an upper region 248, which, for applications where radiation is applied to the tissue surface, would be the epidermis and dermis, and a lower region 250, which would be a subdermal region in the previous example. Region 250, for instance, can be the hypodermis.

FIGS. 1A-1C show another schematic representation of a system 208 for creating islets of treatment. FIGS. 1A-1C show a system for delivering optical radiation to a treatment volume V located at a depth d in the tissue and having an area A. FIGS. 1A-1C also show treatment or target portions 214 (i.e., islets of treatment) in the tissue 200. A portion of a skeletal muscle tissue 200 is shown, which includes an epimysium 206 overlying a portion of a fassicle 204. The treatment volume V may be below the tissue surface 202 in one or more tissue layers or the treatment volume may extend from the tissue surface through one or more tissue layers.

The system 208 of FIGS. 1A-1C can be incorporated within a hand held device. System 208 includes an energy source 210 to produce electromagnetic radiation (EMR). The output from energy source 210 is applied to an optical system 212, which is preferably in the form of a delivery head in contact with the surface of the tissue, as shown in FIG. 1C. The delivery head can include, for example, a contact plate or cooling element 216 that contacts the tissue. The system 208 can also include detectors 216 and controllers 218. The detectors 216 can, for instance, detect contact with the tissue and/or the speed of movement of the device over the tissue and can, for example, image the tissue. The controller 218 can be used, for example, to control the pulsing of an EMR source in relation to contact with the tissue and/or the speed of movement of the hand piece. (Note that throughout this specification, the terms “head”, “hand piece” and “hand held device” may be used interchangeably. Each of these components is discussed in greater detail below.)

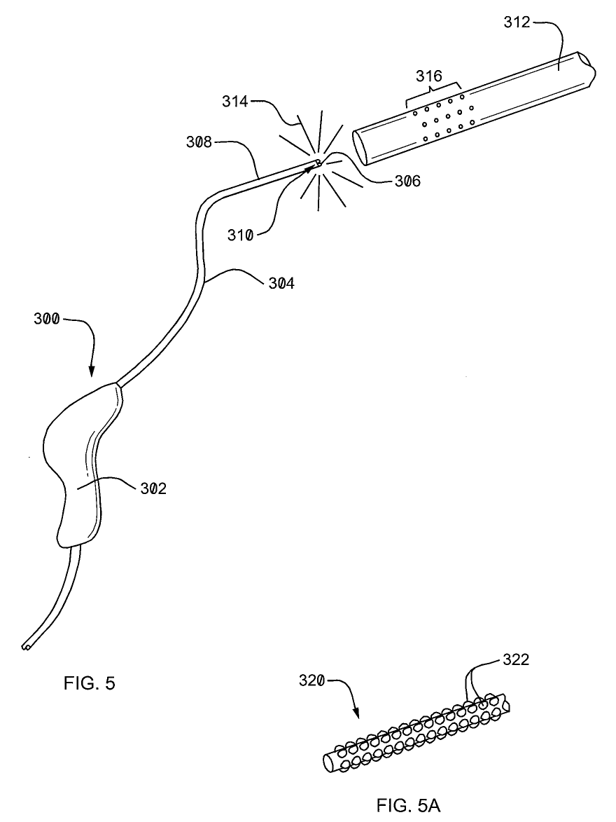

In other embodiments, fiber delivery of laser light using endoscopic methods or arthroscopic scope enable treatments of certain tissues without more extensive surgical procedures. For example, referring to FIG. 5, an endoscope 300 has a handpiece 302 and a flexible tube 304 which contains an internal optical fiber (not shown) that extends from the handpiece to a distal end of the tube 308. Endoscope 300 includes an EMR-transmission mechanism 306 located at the distal end 308 of tube 304 and a motion sensor 310 is located adjacent to EMR-transmission mechanism 306. In the present embodiment, the EMR-transmission mechanism 306 is an array of lenses or other transmitting elements such as optical fibers that are configured to irradiate multiple light beams 314 in a direction normal to the perimeter of tube 304, and motion sensor 310 is an infrared sensor. Many alternate configurations of the EMR-transmission mechanism are possible, however, and will vary depending on the application.

In operation, tube 304 is inserted into a blood vessel 312 (or other lumen or other tissue, such as fat tissue, or organs such as the heart, stomach or other organs of the digestive tract). Once inserted and positioned, the EMR-transmission mechanism 306 is pulled back through and along the length of the vessel to be treated. The motion sensor 310 measures the speed of motion of the EMR-transmission mechanism relative to the internal wall of the blood vessel 312. A controller (not shown) causes EMR to be irradiated from the EMR-transmission mechanism 306 in pulses at a predetermined rate based on the speed that EMR-transmission mechanism 306 travels within the vessel 312. This causes the light beams 314 to be irradiated intermittently along the blood vessel 312 and create a pattern of EMR-treated islets 316 along the blood vessel 312.

In an alternate embodiment shown in FIG. 5A, a tube of an endoscope may be inserted in a tube 320 that contains optically transparent windows 322 through which the EMR-treated islets are created. In this embodiment, tube 320 is sized to fit within vessel 312 such that the windows 322 are in close proximity to the internal walls of the vessel 312. The windows 322 are a set of lenses. Various patterns of lenses are possible as discussed, for example, in conjunction with FIGS. 6A, and 7-8. In still another embodiment, the EMR transmission mechanism can be fashioned similar to tube 320, but be in the form of an integrated end-piece that irradiates EMR through an array of windows similar to windows 322. The EMR-transmission mechanism can be inserted into a blood vessel (or other tissue, lumen or organ) and positioned to create EMR treated islets as needed.

The EMR-treatment mechanism 306 can be gradually moved forward or withdrawn as the pulsations are emitted resulting in an array of islets in the internal wall of the surface spaced according to the repetition rate and velocity of the motion of the device. In this way treatment may be applied to internal structures through an endoscope (or, in alternate embodiments, a catheter or other device) such that more extensive surgical access is not required. Many alternate embodiments are possible and the devices, methods and parameters used will vary with, for example, the treatment being performed, the type of tissue being treated, and the location of the tissue. Examples of other possible treatments include, without limitation, arthroscopic knee surgery, esophageal treatments, stomach and intestinal treatments, muscle and fasciae treatments, carpal tunnel, etc.

Similarly, the technique can also be applied to conventional light-based liposuction treatments. For example, a small cannula, or tube, containing a laser fiber may be inserted into the skin and passed throughout the treatment area. The laser's energy may be applied directly to the fat cells such that EMR-islets are created within the tissue, causing the fat cells to rupture and drain away. Additionally, EMR-islets can be formed, simultaneously or in a separate step or procedure, in surrounding tissue to cause a healing response in the tissue surrounding the fat cells that, for example, will allow the tissue to reform as firm tissue and reduce sagging and other effects from the loss of significant amounts fat cells.

A. Electromagnetic Radiation Sources

The energy source 210 may be any suitable optical energy source, including coherent and non-coherent sources, able to produce optical energy at a desired wavelength or a desired wavelength band or in multiple wavelength bands. The exact energy source 210, and the exact energy chosen, may be a function of the type of treatment to be performed, the tissue to be heated, the depth within the tissue at which treatment is desired, and of the absorption of that energy in the desired area to be treated. For example, energy source 210 may be a radiant lamp, a halogen lamp, an incandescent lamp, an arc lamp, a fluorescent lamp, a light emitting diode, a laser (including diode and fiber lasers), the sun, or other suitable optical energy source. In addition, multiple energy sources may be used which are identical or different. For example, multiple laser sources may be used and they may generate optical energy having the same wavelength or different wavelengths. As another example, multiple lamp sources may be used and they may be filtered to provide the same or different wavelength band or bands. In addition, different types of sources may be included in the same device, for example, mixing both lasers and lamps.

Energy source 210 may produce electromagnetic radiation, such as near infrared or visible light radiation over a broad spectrum, over a limited spectrum, or at a single wavelength, such as would be produced by a light emitting diode or a laser. In certain cases, a narrow spectral source may be preferable, as the wavelength(s) produced by the energy source may be targeted towards a specific tissue type or may be adapted for reaching a selected depth. In other embodiments, a wide spectral source may be preferable, for example, in systems where the wavelength(s) to be applied to the tissue may change, for example, by applying different filters, depending on the application.

Depending on the application, many types of electromagnetic radiation, and other forms of energy in some cases, may be used. For example, UV, violet, blue, green, yellow light or infrared radiation (e.g., about 290-600 nm, 1400-3000 nm) can be used for treatment of superficial targets, such as vascular and fascia. Blue, green, yellow, red and near IR light in a range of about 450 to about 1300 nm can be used for treatment of a target at depths up to about 1 millimeter below the tissue surface. Near infrared light in a range of about 800 to about 1400 nm, about 1500 to about 1800 nm or in a range of about 2050 nm to about 2350 nm can be used for treatment of deeper targets (e.g., up to about 3 millimeters beneath the surface)—(See Table 1B). Additionally, acoustic, RF or other EMF sources may also be employed in suitable applications.

1. Coherent Light Sources.

The energy source 210 can be any variety of a coherent light source, such as a solid-state laser, dye laser, diode laser, fiber laser, or other coherent light source. For example, the energy source 210 can be a neodymium (Nd) laser, such as a Nd:YAG laser. In this exemplary embodiment, the energy source 210 includes a neodymium (Nd) laser generating radiation having a wavelength around 1064 nm. Such a laser includes a lasing medium, e.g., in this embodiment a neodymium YAG laser rod (a YAG host crystal doped with Nd⁺³ ions), and associated optics (e.g., mirrors) that are coupled to the laser rod to form an optical cavity for generating lasing radiation. In other embodiments, other laser sources, such as chromium (Cr), Ytterbium (Yt) or diode lasers, or broadband sources, e.g., lamps, can be employed for generating the treatment radiation.

Lasers and other coherent light sources can be used to cover wavelengths within the 100 to 100,000 nm range. Examples of coherent energy sources are solid state, dye, fiber, and other types of lasers. For example, a solid state laser with lamp or diode pumping can be used. The wavelength generated by such a laser can be in the range of 400-3,500 nm. This range can be extended to 100-20,000 nm by using non-linear frequency converting. Solid state lasers can provide maximum flexibility with pulse width range from femtoseconds to a continuous wave.

Another example of a coherent source is a dye laser with non-coherent or coherent pumping, which provide wavelength-tunable light emission. Dye lasers can utilize a dye dissolved either in liquid or solid matrices. Typical tunable wavelength bands cover 400-1,200 nm and a laser bandwidth of about 0.1-10 nm. Mixtures of different dyes can provide multi wavelength emission. Dye laser conversion efficiency is about 0.1-1% for non-coherent pumping and up to about 80% with coherent pumping. Laser emission may be delivered to the treatment site by an optical waveguide, or, in other embodiments, a plurality of waveguides or laser media may be pumped by a plurality of laser sources (lamps) next to the treatment site. Such dye lasers can result in energy exposure up to several hundreds of J/cm², pulse duration from picoseconds to tens of seconds, and a fill factor from about 0.1% to 90%.

Another example of a coherent source is a fiber laser. Fiber lasers are active waveguides a doped core or undoped core (Raman laser), with coherent or non-coherent pumping. Rare earth metal ions can be used as the doping material. The core and cladding materials can be quartz, glass or ceramic. The core diameter may be from microns to hundreds of microns. Pumping light may be launched into the core through the core facet or through cladding. The light conversion efficiency of such a fiber laser may be up to about 80% and the wavelength range can be from about 1,100 to 3,000 nm. A combination of different rare-earth ions, with or without additional Raman conversion, can provide simultaneous generation of different wavelengths, which may benefit treatment results. The range can be extended with the help of second harmonic generation (SHG) or optical parametric oscillator (OPO) optically connected to the fiber laser output. Fiber lasers can be combined into the bundle or can be used as a single fiber laser. The optical output can be directed to the target with the help of a variety of optical elements described below, or can be directly placed in contact with the tissue with or without a protective/cooling interface window. Such fiber lasers can result in energy exposures of up to about several hundreds of J/cm² and pulse durations from about picoseconds to tens of seconds.

Diode lasers can be used for, e.g., the 400-100,000 nm range. Since many photodermatology applications require a high-power light source, the configurations described below using diode laser bars can be based upon about 10-100 W, 1-cm-long, cw diode laser bar. Note that other sources (e.g., LEDs and microlasers) or embodiments can be designed using lower power (mW to 10 W) sources can be substituted in the configurations described for use with diode laser bars with suitable modifications to the optical and mechanical sub-systems.

Other types of lasers (e.g., gas, excimer, etc.) can also be used.

2. Non-Coherent Light Sources

A variety of non-coherent sources of electromagnetic radiation (e.g., arc lamps, incandescence lamps, halogen lamps, light bulbs) can be used as an energy source. There are several monochromatic lamps available such as, for example, hollow cathode lamps (HCL) and electrodeless discharge lamps (EDL). HCL and EDL may generate emission lines from chemical elements. For example, sodium emits bright yellow light at 550 nm. The output emission may be concentrated on the target with reflectors and concentrators. Energy exposures up to about several tens of J/cm², pulse durations from about picoseconds to tens of seconds, and fill factors of about 1% to 90% can be achieved.

Linear arc lamps use a plasma of noble gases overheated by pulsed electrical discharge as a light source. Commonly used gases are xenon, krypton and their mixtures, in different proportions. The filling pressure can be from about several torr to thousands of torr. The lamp envelope for the linear flash lamp can be made from fused silica, doped silica or glass, or sapphire. The emission bandwidth is about 180-2,500 nm for clear envelope, and may be modified with a proper choice of dopant ions inside the lamp envelope, dielectric coatings on the lamp envelope, absorptive filters, fluorescent converters, or a suitable combination of these approaches.

In some embodiments, a Xenon-filled linear flash lamp with a trapezoidal concentrator made from BK7 glass can be used. As set forth in some embodiments below, the distal end of the optical train can, for example, be an array of microprisms attached to the output face of the concentrator. The spectral range of EMR generated by such a lamp can be about 300-2000 nm, energy exposure can be up to about 1,000 J/cm², and the pulse duration can be from about 0.1 ms to 10 s.

Incandescent lamps are one of the most common light sources and have an emission band from 300 to 4,000 nm at a filament temperature of about 2,500 C. The output emission can be concentrated on the target with reflectors and/or concentrators. Incandescent lamps can achieve energy exposures of up to about several hundreds of J/cm² and pulse durations from about seconds to tens of seconds.

Halogen tungsten lamps utilize the halogen cycle to extend the lifetime of the lamp and permit it to operate at an elevated filament temperature (up to about 3,500 C), which greatly improves optical output. The emission band of such a lamp is in the range of about 300 to 3,000 nm. The output emission can be concentrated on the target with reflectors and/or concentrators. Such lamps can achieve energy exposures of up to thousand of J/cm² and pulse durations from about 0.2 seconds to continuous emission.

Light-emitting diodes (LEDs) that emit light in the 290-2,000 nm range can be used to cover wavelengths not directly accessible by diode lasers.

Referring again to FIGS. 1A-1C, the energy source 210 or the optical system 212 can include any suitable filter able to select, or at least partially select, certain wavelengths or wavelength bands from energy source 210. In certain types of filters, the filter may block a specific set of wavelengths. It is also possible that undesired wavelengths in the energy from energy source 210 may be wavelength shifted in ways known in the art so as to enhance the energy available in the desired wavelength bands. Thus, filter may include elements designed to absorb, reflect or alter certain wavelengths of electromagnetic radiation. For example, filter may be used to remove certain types of wavelengths that are absorbed by surrounding tissues or hemoglobin. For instance, many internal tissues are primarily composed of water, such as most organs and much of the rest of the human body. By using a filter that selectively removes wavelengths that excite water molecules, the absorption of these wavelengths by the body may be greatly reduced, which may contribute to a reduction in the amount of heat generated by light absorption in these molecules. Thus, by passing radiation through a water-based filter, those frequencies of radiation that may excite water molecules will be absorbed in the water filter, and will not be transmitted into tissue. Thus, a water-based filter may be used to decrease the amount of radiation absorbed in tissue above the treatment region and converted into heat. For other treatments, absorption of the radiation by water may be desired or required for treatment. In another instance, such as a vessel wall treatment, absorption by oxy- or deoxy-hemoglobin may be substancially reduced by using a filter that selectively removes wavelengths with high absorption in these molecules and enable treatment of the vessel wall with less absorption of radiation by the internal fluid.

B. Optical System

Generally, optical system 212 of FIGS. 1A-1C functions to receive radiation from the source 210 and to focus or concentrate such radiation to one or more beams 222 directed to a selected one or more treatment or target portions 214 of volume V, the focus being both to the depth d and spatially in the area A (see FIG. 1C). Some embodiments use such an optical system 212, and other embodiments do not use an optical system 212. In some embodiments, the optical system 212 creates one or more beams which are not focused or divergent. In embodiments with multiple sources, optical system 212 may focus/concentrate the energy from each source into one or more beams and each such beam may include only the energy from one source or a combination of energy from multiple sources.

If an optical system 212 is used, the energy of the applied light can be concentrated to deliver more energy to target portions 214. Depending on system parameters, portions 214 may have various shapes and depths as described above.

The optical system 212 as shown in FIGS. 1A-1C may focus energy on portions 214 or a selected subset of portions 214 simultaneously. Alternatively, the optical system 212 may contain an optical or mechanical-optical scanner for moving radiation focused to depth d to successive portions 214. In another alternative embodiment, the optical system 212 may generate an output focused to depth d and may be physically moved on the tissue surface over volume V, either manually or by a suitable two-dimensional or three-dimensional (including depth) positioning mechanism, to direct radiation to desired successive portions 214. For the two later embodiments, the movement may be directly from portion to portion to be focused on or the movement may be in a standard predetermined pattern, for example a grid, spiral or other pattern, with the EMR source being fired only when over a desired portion 214.

Where an acoustic, RF or other non-optical EMR source is used as energy source 210, the optical system 212 can be a suitable system for concentrating or focusing such EMR, for example a phased array, and the term “optical system” should be interpreted, where appropriate, to include such a system.

C. Accessory Elements for Cooling, Heating, Reflecting, Absorbing, Blocking as Ancillary Process to Guide, Restrict or Modify Effects of Radiation on Tissue.

As discussed above, the system 208 can also include a cooling element 215 to cool the surface of the tissue 200 over treatment volume V. As shown in FIGS. 1A-1C, a cooling element 215 can act on the optical system 212 to cool the portion of this system in contact with the tissue, and thus the portion of the tissue in contact with such element. In some embodiments intended for use on fascia or other thin tissues, the cooling element 215 might not be used or, alternatively, might not be cooled during treatment (e.g., cooling only applied before and/or after treatment). In some embodiments, cooling can be applied fractionally on a portion of the tissue surface (cooling islets), for example, between optical islets. In some embodiments, cooling of the tissue is not required and a cooling element might not be present on the hand piece. In other embodiments, cooling may be applied only to the portions of tissue between the treatment islets in order to increase contrast.

Treatment of internal tissues of the body during may be manipulated so that the cooling element is applied to the internal side of the tissue or through liquid perfused through the tissue. In these embodiments, cooling may be used to control the depth of effective treatment by preventing the internal surfaces from reaching temperatures that are damaging. When treating a vessel, for instance, one way to protect the inner vessel wall surface would be to perfuse or prefill the vessel with cooled fluid such as saline, lactated ringers, or blood plasma during application of radiation to the external wall surface as part of treatment.

In alternate embodiments, materials may be externally applied to facilitate treatment. For example, heat, radiation absorptive material, and/or reflective material may be used to guide, direct, restrict and focus energy to a target tissue or prevent exposure to another adjacent tissue. A heated radiation absorptive or reflective surface may be used to enhance depth of penetration or extent of tissue action. In some applications, the tissue may be preheated or precooled to a set point temperature to enable treatment at a specific and/or predetermined target depth.

Similarly, EMR can be applied from two or more different locations during the treatment, such as from two sides of a muscle, blood vessel or other tissue or from within and without an organ or blood vessel or from locations internal and external to a body. Such treatments may serve various functions. For example, in one embodiment, EMR can be applied to two sides of (or from two locations within) a muscle, organ wall or other tissue using parameters that are selected such that EMR from each individual location does not cause the formation of EMR-treated islets standing alone, but that does create EMR-treated islets within the muscle, organ wall or other tissue throughout a volume of tissue where the EMR from the two locations converges and/or overlaps at a sufficient intensity to cause the formation of EMR-treated islets. The parameters may be chosen to not cause the formation of EMR-treated islets at the surface of the tissue. Alternatively, the parameters could be chosen to treat the entire volume between the locations where EMR is applied, including at any surface of the tissue. The later case may be used, for example, if the treatment would benefit from irradiating the tissue from one or more sides or locations to create a more uniform intensity and/or dispersion of EMR throughout the tissue volume. One skilled in the art will further appreciate that such a technique could also be applied without forming EMR-treated islets and instead treating a relatively larger contiguous volume of tissue or treating only a single relatively small tissue volume.

The cooling (or blocking, reflecting or heating) element 215 can include a system for cooling (blocking, reflecting or heating) the optical system (and hence the portion in contact with the tissue) as well as a contact plate that touches the tissue when in use. The contact plate can be, for example, a flat plate, a series of conducting pipes, a sheathing blanket, or a series of channels for the passage of air, water, oil or other fluids or gases. Mixtures of these substances may also be used. For example, in one embodiment, the cooling system can be a water-cooled contact plate or ring. The cooling mechanism may be a plate and may also include a series of channels carrying a coolant fluid or a refrigerant fluid (for example, a cryogen), which channels are in contact with a plate that is in contact with the tissue. In yet another embodiment, the cooling system may comprise a water or refrigerant fluid (for example R134A) spray, a cool air spray or air flow across the surface of the tissue. In other embodiments, cooling may be accomplished through chemical reactions (for example, endothermic reactions), or through electronic cooling, such as Peltier cooling.

In certain cases, cooling mechanism may be used to maintain the surface temperature of the tissue at its normal temperature, which may be, for example, 37° C., but will vary depending on the type of tissue being heated. In other embodiments, cooling mechanism may be used to decrease the temperature of the surface of the tissue to a temperature below the normal temperature of that type of tissue. For example, the cooling mechanism may be able to decrease the surface temperature of tissue to, for example, a range between 25° C. and −5° C. In other embodiments, a plate can function as a heating plate in order to heat the tissue. Some embodiments can include a plate that can be used for cooling and heating.

A contact plate of the cooling element may be made out of a suitable heat transfer material, and also, where the plate contacts the tissue, of a material having a good optical match with the tissue. Sapphire is an example of a suitable material for the contact plate. Where the contact plate has a high degree of thermal conductivity, it may allow cooling of the surface of the tissue by the cooling mechanism. In other embodiments, contact plate may be an integral part of cooling mechanism, or may be absent. In some embodiments, such as shown in FIGS. 1A-1C, energy from energy source 210 may pass through contact plate. In these configurations, contact plate may be constructed out of materials able to transmit at least a portion of energy, for example, glass, sapphire, or a clear plastic. In addition, the contact plate may be constructed in such a way as to allow only a portion of energy to pass through contact plate, for example, via a series of holes, passages, apertures in a mask, lenses, etc. within the contact plate. In other embodiments, energy may not be directed through the cooling mechanism 215.

D. Devices for Producing a Multiplicity of Treated Islets

A number of different devices and structures can be used to spatially modulate and/or concentrate EMR in order to generate islets of treatment in the tissue. For example, the devices can use reflection, refraction, interference, diffraction, and deflection of incident light to create treatment islets. A number of these devices are briefly summarized below, with a more detailed explanation of the devices in the remainder of the specification, and in particular in connection with the section entitled Devices and Systems for Producing Islets of Treatment, Example 4. Methods for generating islets of treatment, and numerous other devices and methods for creating islets of treatment are set forth throughout this specification. In addition, although some devices and methods for generating islets of treatment are briefly set forth below, the invention is not limited to these particular methods and devices.

Splitting of EMR by reflection of the light can be obtained using specular or diffuse reflection of the light from surfaces with refractive indices higher than 1. Splitting of EMR by refraction can be obtained using refraction on angular or curved surfaces. Diffraction splitting is based on the fact that light can bend around edges. Deflection splitting can be achieved when light propagates inside a media with a non-even distribution of refractive indices.

1. Blocking Portions of the EMR Beam

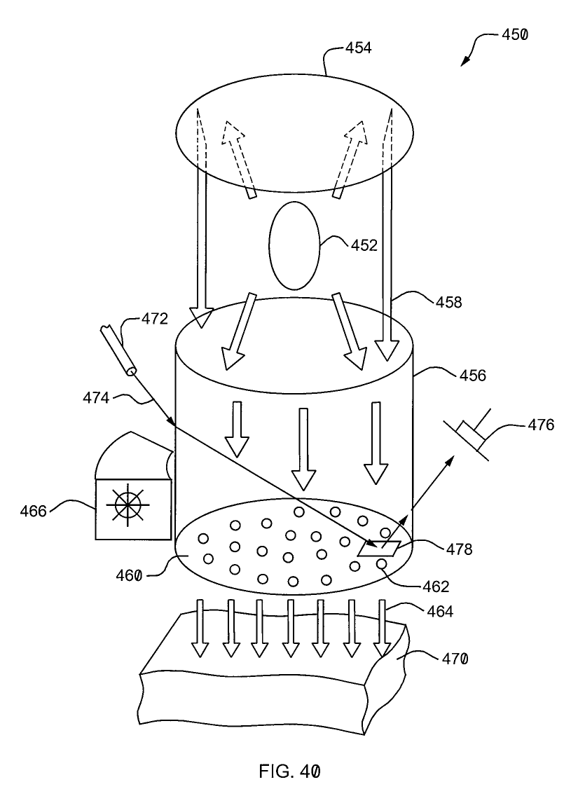

In some embodiments, a mask can be used to block portions of the EMR generated by the EMR source from reaching the tissue. The mask can contain a number of holes, lines, or slits, which function to spatially modulate the EMR to create islets of treatment. FIGS. 39 and 40 illustrate two embodiments in which the islets of treatment are formed generally through the use of a mirror containing holes or other transmissive portions. Light passes through the holes in the mirror and strikes the tissue, creating islets of treatment. Light reflected by the mirror stays in the optical system through a system of reflectors and may be redirected through the holes to improve efficiency. One effective mask is a contact cooling mask (i.e., it contacts the tissue during treatment) with a high reflection and minimum absorption for masking light.

2. Focusing, Directing, or Concentrating the EMR Beam

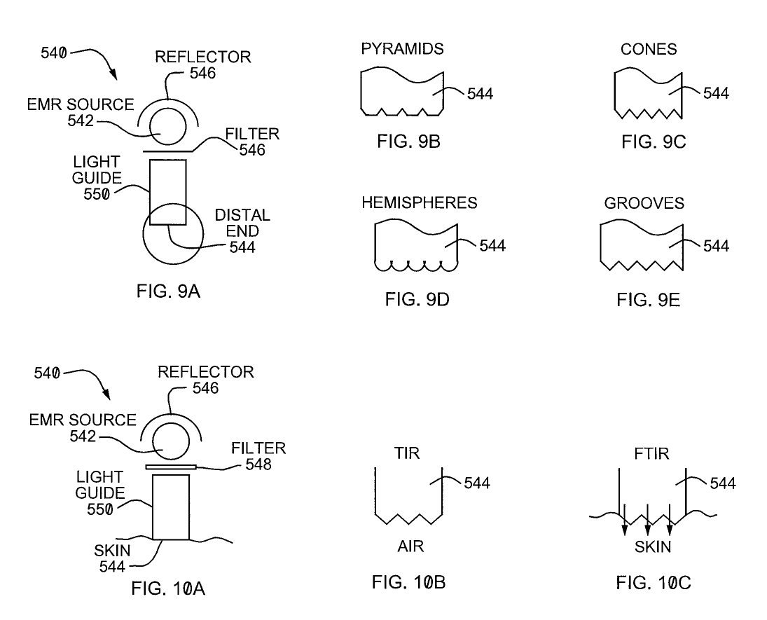

In some embodiments, spatial modulation and concentration of the EMR can be achieved by shaping an end portion of a light guide with prisms, pyramids, cones, grooves, hemispheres, or the like in order to create output light spatial modulation and concentration, and therefore to form islets of treatment in a tissue. For example, FIGS. 9A through 10A depict such embodiments. Numerous exemplary types of imaging optics and/or diffractive optics can also be used in this embodiment.

In addition, in some embodiments, such as that of FIG. 1A-10C, the end of the light guide can be shaped in order to introduce light total internal reflection (TIR) when the distal end of the device is in contact with air, while allowing EMR to pass through when the distal end is in contact with a lotion or tissue surface.

Alternatively, some embodiments can use spatially modulated phase arrays to introduce phase shifts between different portions of the incident beam. As a result of interference between the said portions, amplitude modulation is introduced in the output beam.

3. Arrays of EMR Sources

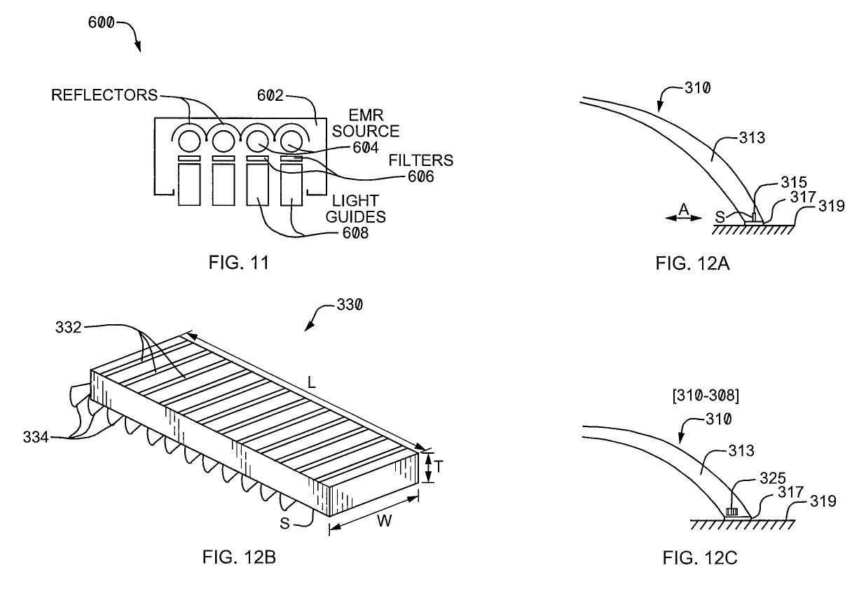

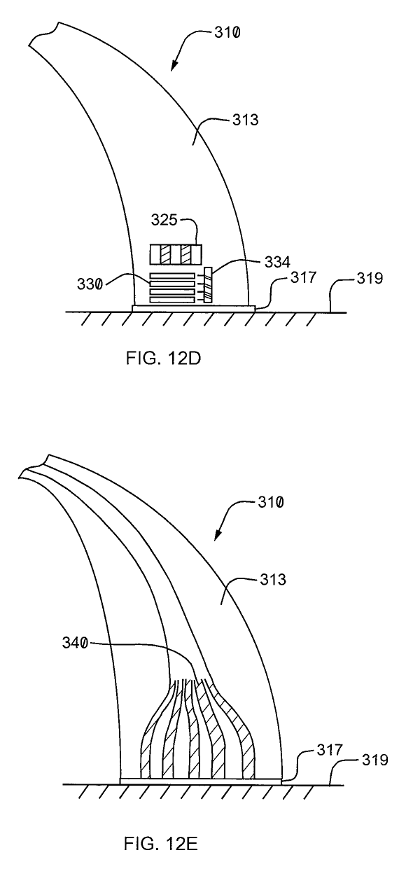

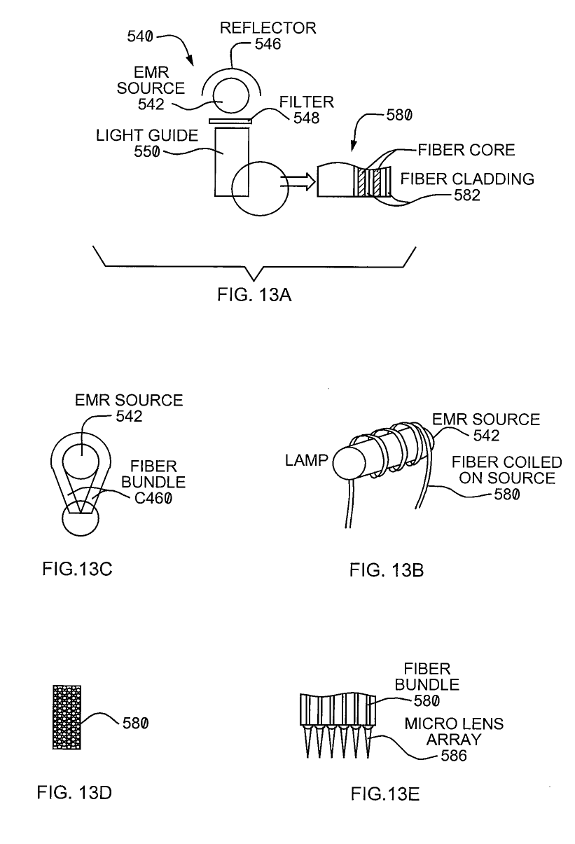

Instead of splitting the EMR into multiple beams, one can use a plurality of light sources or a single light source with a serial or parallel optical multiplexer to form islets of treatment in the tissue. For example, the embodiment of FIG. 11 uses a line or array of non-coherent EMR sources to create islets of treatment. Other embodiments, such as that shown in FIG. 12C, use an array of diode laser bars in order to form islets of treatment. Still other embodiments, use a bundle of optical fibers to deliver spatially modulated EMR to the tissue. FIGS. 12E, 13B-D, and 14A are exemplary embodiments that use a bundle of optical fibers.

4. Pulsing the EMR Source

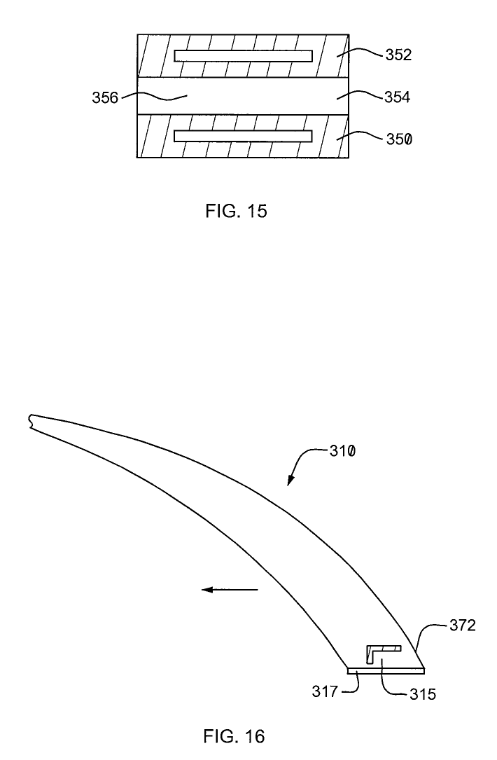

Some embodiments can include a sensor for determining the speed of movement of the hand piece across the target area of the tissue. The hand piece can further include circuitry in communication with the sensor for controlling the optical energy in order to create islets of treatment. The circuitry can control, for example, pulsing of the optical energy source based on the speed of movement of the head portion across the tissue in order to create islets of treatment. In another embodiment, the circuitry can control movement of the energy source, a scanner or other mechanism within the apparatus based on the speed of movement of the head portion across the tissue in order to expose only certain areas of the tissue to the EMR energy as the head is moved over the tissue in order to create islets of treatment. FIGS. 15 and 16 are exemplary embodiments according to this aspect.

5. Lattices of Exogenous Chromophores

In other embodiments, spatially selective islets of treatment can be created by applying to the tissue surface a desired pattern of a topical composition containing a preferentially absorbing exogenous chromophore. The chromophore can also be introduced into the tissue with a needle, for example, a micro needle as used for tattoos. In this case, the EMR energy may illuminate the entire tissue surface where such pattern of topical composition has been applied. Upon application of appropriate EMR, the chromophores can heat up, thus creating islets of treatment in the tissue. Alternatively, the EMR energy may be focused on the pattern of topical composition. A variety of substances can be used as chromophores including, but not limited to, carbon, metals (Au, Ag, Fe, etc.), organic dyes (Methylene Blue, Toluidine Blue, etc.), non-organic pigments, nanoparticles (such as fullerenes), nanoparticles with a shell, carbon fibers, etc. The desired pattern can be random and need not be regular or pre-determined. It can vary as a function of the tissue condition at the desired treatment area and be generated ad hoc.

Some embodiments provide a film or substrate material with a lattice of dots, lines or other shapes, either on the surface of the film or embedded within the film, in which the dots, lines or other shapes include a chromophore appropriate to the EMR source. The dots, lines or other shapes may be the same or different sizes and different shapes may be included on the film.

6. Creating Thermal Lattices Using Patterned Cooling

Some embodiments can produce thermal (and damage) lattices (or treatment islets) by employing uniform EMR beams and spatially modulated cooling devices. The resulting thermal lattice in such cases will be inverted with respect to the original cooling matrix.

7. Creating, Blocking or Facilitating Patterned Treatment Through Perfusion of Tissue with Chromophore or Other Radiation Manipulative Chemical Material.

Some embodiments for treatment internal tissue may use associative agents to target light to specific cells or aspects of the tissue by pretreating tissue or applications to the tissue during treatment. Dyes or other such agents can be used to absorb or protect vessels within a target tissue.

E. Controllers and Feedback Systems

Some embodiments can also include speed sensors, contact sensors, imaging arrays, and controllers to aid in various functions of applying EMR to the tissue. System 208 of FIG. 1A-1B includes an optional detector 216, which may be, for example, a capacitive imaging array, a CCD camera, a photodetector, or other suitable detector for a selected characteristic of the tissue. The output from detector 216 can be applied to a controller 218, which is typically a suitably programmed microprocessor or other such circuitry, but may be special purpose hardware or a hybrid of hardware and software. Control 218 can, for example, control the turning on and turning off of the light source 210 or other mechanism for exposing the light to the tissue (e.g., shutter), and control 218 may also control the power profile of the radiation. Controller 218 can also be used, for example, to control the focus depth for the optical system 212 and to control the portion or portions 214 to which radiation is focused/concentrated at any given time. Finally, controller 218 can be used to control the cooling element 215 to control both the tissue temperature above the volume V and the cooling duration, both for pre-cooling and during irradiation.

In other embodiments, real time acquired images can be used for statistical analysis of, for example, a marker concentration in an exogenous substance or for other purposes associated with the procedure. Images can also be used to visualize tissue that it out of the line of sight of a surgeon during a procedure. The imaging system can also function as a contact sensor. This allows for real time determination of immediate contact of a hand piece with the tissue. The combination of hardware and software allows this determination within one image frame. The algorithm measures in real time a tissue contact and navigation parameters (position, velocity and acceleration) along the x-axis and y-axis. A capacitive sensor along with image processing and special lotion can be used for detecting tissue imperfections and measuring the size of the imperfection in real time. The resolution of the sensor will depend on pixel size, image processing and the sub pixel sampling.

The capacitive sensor and image processing also allow for determination of whether the device is operating on biological tissue or some form of other surface. It is possible under proper sampling conditions to extract the type of tissue the device is moving across.

F. Creation of Lattices Using Non-Optical EMR Sources

The lattices can also be produced using non-optical sources. For example, as noted above, microwave, radio frequency and low frequency or DC EMR sources can be used as energy sources to create lattices of EMR-treated islets. In addition, for treating tissue surfaces, the tissue surface can be directly contacted with heating elements in the pattern of the desired lattice.

The following examples illustrate some preferred modes of practicing some of the embodiments, but are not intended to limit the scope of the claimed invention. Alternative parameters, materials, methods and devices may be utilized to obtain similar, additional or other results.

B. Theoretical Model of Islet Lattice Relaxation

The theory of selective photothermolysis considers the thermal relaxation time (TRT) of an individual target as the characteristic time required for an overheated target to come to the thermal equilibrium with its environment. It is suggested that the TRT is d²/(8α), d²/(16α), and d²/(24α) for the planar (one-dimensional), cylindrical (two-dimensional), and spherical (three-dimensional) targets, with d being the target width (one-dimensional) or diameter (two or three-dimensional).

This definition can be extended to an islet lattice. Significantly, if the lattice is very sparse, i.e., the fill factor is much smaller than 1, the LTRT can be almost equal to the TRT of an individual islet. It can be expected, however, that dense lattices will come to an equilibrium faster than the sparse ones, as well as that the LTRT will be determined predominantly by the dimensionality of the lattice, its fill factor, and the islet TRT.

A precise definition of LTRT was formulated as follows: let the islets be heated to temperature T₀ at time zero with the tissue temperature in between them being T_{b}<T₀. If no external action occurs, the temperature gradients in the lattice will decay in time and the lattice will approach the thermal equilibrium at stationary temperature Tₛₜ=T_{b}+(T₀−T_{b})·f. Since the stationary temperature cannot be reached for a finite time, the LTRT can be defined as the time needed for the islets to cool down to the intermediate temperature

$${T}_{1} = {T}_{st} + ( {T}_{0} - {T}_{st} ) \cdot {e}^{- 1} = {T}_{b} + ( {T}_{0} - {T}_{b} ) \cdot \frac{1 + f \cdot ( e - 1 )}{e} ,$$

with e being the natural logarithm base.

The LTRT is dependent on the lattice fill factor, f which can be illustrated by first considering the particular case of the two-dimensional lattice. Disregarding the effect of the precise voxel and islet shapes, it can be assumed that the islet and the voxel are infinite cylinders of radii r₀ and R=r₀/\sqrt{f}, respectively. Apparently, the cylindrical pattern cannot be translated in space to form a lattice. However, it is unlikely that the transformation of the actual voxel into the cylinder of the same cross-sectional area can change the LTRT appreciably. The significance of this transformation is that it decreases the dimensionality of the problem to 1. The time-dependent heat equation within the cylindrical voxel was solved mathematically by applying a periodic (symmetry) boundary conditions on its outer surface.

Therefore, the heat equation, the initial condition, and the boundary conditions in the cylindrical frame can be written as follows:

$$\begin{matrix}ρ c \frac{\partial }{\partial t} T ( r , t ) = \frac{κ}{r} \frac{\partial }{\partial r} ( r \frac{\partial }{\partial r} T ( r , t ) ) , & ( A12 ) \\ T ( r , 0 ) = {T}_{0} \cdot \{ \begin{matrix}1 , & r \leq {r}_{0} , \\ 0 , & r > {r}_{0} ,\end{matrix} & ( A13 ) \\ \frac{\partial }{\partial t} T ( 0 , t ) = \frac{\partial }{\partial t} T ( R , t ) = 0 , & ( A14 )\end{matrix}$$

where ρ, c, and κ are the density, the specific heat, and the thermal conductivity of the tissue. It is suggested that T_{b}=0, which does not limit the generality of the analysis. Introducing the dimensionless time τ=t/TRT and the dimensionless coordinate ξ=r/r₀ (where TRT=d₀²/(16α)=r₀²/(4α) is the TRT of the cylindrical islet and α=κ/(ρc) is the thermal diffusivity) the following equations were obtained:

$$\begin{matrix}\frac{\partial }{\partial τ} T ( ξ , τ ) = \frac{1}{4 ξ} \frac{\partial }{\partial ξ} ( ξ \frac{\partial }{\partial ξ} T ( ξ , τ ) ) , & ( A15 ) \\ T ( ξ , 0 ) = {T}_{0} \cdot \{ \begin{matrix}1 , & ξ \leq 1 , \\ 0 , & ξ > 1 ,\end{matrix} & ( A16 ) \\ \frac{\partial }{\partial τ} T ( 0 , τ ) = \frac{\partial }{\partial τ} T ( \sqrt{{f}^{- 1}} , τ ) = 0. & ( A17 )\end{matrix}$$

Equations (A15)-(A17) can be solved numerically to evaluate the LTRT, that is the time when the temperature at the voxel center reduces to

$$T ( 0 , τ ) = {T}_{1} = {T}_{0} \cdot \frac{f + 1}{2} .$$

It is worth noting that set (A15)-(A17) is linear with respect to temperature and the LTRT does not depend on the initial temperature thereof. Consequently, the ratio of the LTRT to the islet TRT depends on the lattice fill factor only. Apparently, this simplification comes from the assumptions made for reducing the dimensionality of the problem.

C. Lattice Temperature Relaxation Time

To obtain the lattices of the thermal islets (LTI), a corresponding lattice of optical islets (LOI) has to be created first. The next step is to make the pulse width short enough to avoid overlapping of the adjacent thermal islets. It should be emphasized that LTI is a time-dependent structure and the latter requirement implies that the islets should not overlap at the time instant when the temperature reaches its maximum.

The limitation on the pulse width may be specified in the context of the theory of selective photothermolysis (Anderson et al. (1983), Science 220: 524-26; Altshuler et al. (2001), Lasers in Surgery and Medicine 29: 416-32). In its original formulation this theory deals with isolated targets inside tissue. It points out that the selective heating of a target is possible if the pulse width is smaller than some time interval characteristic for the target and referred to as the temperature relaxation time (TRT). The TRT, in essence, is the cooling time of the target, which is the time required by an instantly heated target to cool to 1/e of its initial temperature. This concept is applicable easily to the individual islets. It may be pointed out that the TRT of the planar islet (layer of the tissue, one-dimensional) is d²/(8α) with d and α being the target width and the thermal diffusivity of the tissue, respectively. For the cylindrical (two-dimensional) and spherical (three-dimensional) targets the corresponding relations read: d²/(16α) and d²/(24α) with d being the islet diameter (Altshuler et al. (2001), Lasers in Surgery and Medicine 29: 416-32). This concept was generalized to periodic lattices of the optical islets as discussed below.

It is postulated that the lattice temperature dynamics depends on the relation between the islet and voxel areas rather than by the precise islet and voxel shapes. This should be valid if the voxels are not very anisotropic, i.e., long in one direction and short in the others. The anisotropic lattices, in turn, may be considered as the lattices of smaller dimensionality. In particular, the lattice dimensionality is reduced from 2 to 1 if the voxels are very long and narrow rectangles: it is possible to switch from such rectangles to the infinitely long stripes of the same width making up a one-dimensional lattice.

Thermal dynamics of LTI depends on the method of the LOI introduction into the tissue. First method is a “sequential method” or “sequential LOI”. In this case in every time instant just one (or several distant) optical islet is being created in the tissue. Laser beam scanners can be used to create sequential LOI. Second method is “parallel method” or “parallel LOI”. In this case, a multitude of optical islets are created in the tissue simultaneously during the optical pulse. Thermal interaction between islets in the sequential LOI is minimal. For parallel LOI, thermal interaction between different islets can be very significant. To evaluate the lattice thermal relaxation time (LTRT), for parallel LOI, the same reasoning used to find the TRT of an individual islet is followed. The islets are heated instantly to temperature T₀ keeping the space outside them at the constant background temperature T_{b}<T₀. By letting the islets cool through the conduction of heat to the surrounding tissue, the lattice will approach thermal equilibrium at the stationary temperature

Tₛₜ=T_{b}+(T₀−T_{b})·f, (A22)

which depends on the fill factor. The LTRT may be defined as the characteristic cooling time when the islet temperature (more precisely, the maximum temperature within the islet) reaches the intermediate value between the initial and stationary temperatures:

$${T}_{1} = {T}_{st} + ( {T}_{0} - {T}_{st} ) \cdot {e}^{- 1} = {T}_{b} + ( {T}_{0} - {T}_{b} ) \cdot \frac{1 + f \cdot ( e - 1 )}{e} . \qquad ( A23 )$$

Using this definition the LTRT of a very sparse lattice equals the TRT of an individual islet. For such a lattice each islet cools independently on the others. For denser lattices, however, the temperature profiles from different islets overlap causing the LTRT to decrease. This cooperative effect was studied by evaluating the LTRT to TRT ratio as a function of the fill factor for the particular case of the lattice of the cylindrical islets, as described herein. The LTRT decreases monotonically with the growth of the fill factor. Therefore, the denser is the islet lattice the smaller is the time while the lattice relaxes by coming down to the thermal equilibrium with the surrounding tissue. When the fill factor approaches unity, the LTRT approaches some limit close but somewhat larger than the TRT. The distinction is due to some disagreement between the definition of LTRT used here and the conventional definition of TRT. The real temperature decay is not exponential due to the heating of the surrounding tissues. Therefore, the time necessary for the target to decrease its temperature to 1/e of its initial value is always larger than TRT and this time is the actual upper limit of LTRT (the LTRT approaches this limit when the fill factor is zero).

As a rough estimate of the dependence of the LTRT to TRT ratio on the fill factor, a simple relation may be used:

$$\frac{LTRT}{TRT} \approx \frac{1}{3 \cdot f} , \qquad ( A24 )$$

providing a good fit of the numeric data for f>0.1. Actually, equation (A24) means that the LTRT is proportional to the time interval, Δ²/(2·α), while the heat front covers the distance between the islets Δ=d/\sqrt{f}. If the voxel size is very large compared to the islet diameter, the contrast of the thermal lattice may become small before the heat front covers distance Δ. Therefore, equation (A24) overestimates the LTRT appreciably if f<0.1.

D. Light Fluence Parameters for Islet Formation in a Tissue

In order to get isolated islets, the incident fluence has to be bounded from both above and below: Fₘᵢₙ<F<Fₘₐₓ. The meaning of the latter expression is that the fluence has to be large enough to provide the desired effect within the islets but should be insufficient to cause the same effect in the whole bulk of the tissue. Practically, the right-hand-side inequality is sufficient to avoid the bulk effect in all cases while the left-hand-side warrants the formation of the islets only if the pulse width is rather short so that the relation between the delivered light energy and the attained effect is local. This means that the effect depends on the total irradiance at the same point of the tissue rather than on the average irradiance over some area. For the longer pulses, however, the dependence may become non-local due to the heat and mass transfer within the tissue (Sekins et al. (1990) In Therapeutic Heat and Cold, 4-th edition Ed. Lehmann (Baltimore: Williams & Wilkins) pp. 62-112). Therefore, the islets may not appear even if the left-hand-side inequality holds. Fₘᵢₙ can be found as a fluence needed to heat up tissue in a islet to the threshold temperature for the tissue coagulation, Tₜᵣ. If the pulse width is short enough to neglect the heat conduction, the threshold fluence for the protein coagulation is given by:

Fₘᵢₙ=ρc(Tₜᵣ−Tᵢ)/μₐ, (A18)

where ρ is the tissue density, c is its specific heat, μₐ is the tissue absorption coefficient, and Tᵢ is the initial temperature. The threshold of the bulk damage Fₘₐₓ is the fluence needed to heat up tissue, both within the islets and between the islets (bulk tissue), to the threshold temperature. Because the volume of this tissue is 1/f times larger than the volume occupied by islets:

Fₘₐₓ=Fₘᵢₙ/f (A19)

This formula is based on the assumption that the treatment is safe provided that enough intact tissue is left between the islets for assured recovery. A more conservative assumption is that, in addition to the first criterion, the treatment is safe until the temperature in the islets reaches the threshold of thermomechanical effects, Tₘₐₓ. In this case

Fₘₐₓ=Fₘᵢₙ·(Tₘₐₓ−Tᵢ)/(Tₜᵣ−Tᵢ) (A20)

The first criterion predicts a significant safety gap. For example, for f=0.25, the islets and spaces between them have equal safety margins, Fₘₐₓ/Fₘᵢₙ=4. The second criterion is more restrictive. For tissue, Tₘₐₓ can be determined as the temperature of vaporization of water Tₘₐₓ=100° C. Protein coagulation temperature for ms range pulse width is Tₜᵣ=67° C. and the second criterion yields the safety margin Fₘₐₓ/Fₘᵢₙ=2.1.

Isolated islets are considered before the islet lattices. A typical method of creating a 3-dimensional (three-dimensional) optical islet is focusing light inside the tissue. The optical islet of a high contrast may be obtained if the numerical aperture (NA) of the input beam is sufficiently large. However, if the NA is too large one may expect trapping and waveguide propagation of light in superficial layers of the tissue, which may have a higher or different index of refraction than the underlying tissue.

H. Monte-Carlo Simulations of Light Transport.

The plane or cylindrical optical islets perpendicular to the tissue surface may be obtained by using a narrow collimated light beam in the tissue. A beam is considered collimated in the tissue if it neither converges nor diverges in a non-scattering space with the refractive index matching that of tissue at the depth of treatment zₒ. Minimal diameter of collimated beam can be found from the formula (Yariv (1989) Quantum Electronics (NY: John Wiley and Sons)):

dₘᵢₙ=5(z₀λ/π)^{1/2}, (A21)

where λ is the wavelength. For typical depth zₒ=1 mm and λ=1500 nm, dₘᵢₙ=0.1 mm. The spot profile may be a line (stripe) for the one-dimensional islet and some limited shape like circle or square for the two-dimensional islet. For a circular optical beam (wavelength 1200 nm) of diameter 100 μm striking the tissue through sapphire, the transverse intensity profile of the beam is flat at small depths and transfers to a Gaussian when moving deeper into the tissue. Therefore, the optical islet is a cylinder very sharp at the top and somewhat blurred at the bottom. It will be demonstrated below that the weak irradiance outside the original cylinder may not contribute to the tissue damage provided the pulse is short enough. This opens the opportunity of creating the damage islets of a very precise cylindrical shape.

I. Effects of Beam Diameter and Wavelength on Penetration Depth.

To evaluate the shape of an islet it is important to account for an effect of beam diameter on the penetration depth of light into the tissue. The penetration depth is defined as the depth into the tissue where the irradiance is 1/e of the fluence incident onto the tissue surface. This effect is well studied for beams wider than, typically, 1 mm (Klavuhn (2000) Illumination geometry: the importance of laser beam spatial characteristics Laser hair removal technical note No 2 (Published by Lumenis Inc)). However, if the beam is only several tens of micrometers in diameter, which is much smaller than the diffuse length of light in the tissue, the propagation dynamics may be very different from that of wider beams. In particular, for such narrow beams the irradiance decreases monotonically when moving deeper into the tissue along the beam axis whereas for the wider beams a subsurface irradiance maximum may occur. It should be noted herewith that the total bulk irradiance in tissue is the sum of the direct and scattered components and the subsurface maximum is due to the scattered component only. When the beam diameter decreases the on-axis irradiance becomes predominantly due to the direct component and the subsurface maximum disappears.

The dependence of wavelength to penetration depth appears to be rather flat in contrast to the case of the wide beam (Jacques et al. (1995) In Optical-thermal response of laser-irradiated tissue eds. Welch et al. (NY and London: Plenum Press) pp. 561-606; Jacques (1996) In Advances in Optical Imaging and Photon Migration eds. Alfano et al. 2: 364-71; Anderson et al. (1994), Proc. SPIE MS-102: 29-35). The maximum variation of the penetration depth in the specified range is 30-35% only. The penetration depth is limited by the water absorption and the tissue scattering. Apparently, the effect of scattering is stronger for the narrow beams than for the wide ones. The tissue scattering becomes smaller with the wavelength rise while the water absorption increases. These two effects partially compensate each other and the net variations of the penetration depth are rather small.

J. Dynamics of Damage Development.

The lattices of the damage islets develop from those of the thermal islets. The dynamics of the damage development is thought to be governed by the Arrhenius formula. The relationship between the temperature and damage islets is not straightforward. Various tissue sites may show the same peak temperature but a different damage degree, depending on the time the temperature is maintained above the activation threshold of the coagulation or other desired reaction. Moreover, if the pulse width is small the temperature islets can become very sharp at the end of the pulse. If this is the case, the steep temperature gradients may cause the islets to extend and damage the surrounding tissue after the light is off. The effect of such extension leads to onset of bulk damage when the fill factor increases beyond the safe limit.

The LOI technique has several fundamental differences and potential advantages vs. traditional treatment, which employs uniform optical beams for bulk tissue heating and damage. The following conclusions were reached from the computational and theoretical models of islets and islet formation:

(1) In addition to traditional parameters characterizing light treatment, such as the wavelength, the fluence, the pulse width and the spot size, two new important factors are introduced: the fill factor (fractional volume) and the size of islets. Furthermore, the resulting therapeutic effect can be influenced by the geometry (shape, symmetry) and dimensionality of the lattice and islets. LOI can be introduced at different depths at the tissue. For example, in the tissue LOI can be localized in targeted or selective layers of the tissue and surrounding area. For deep LOI, a focusing technique and selective superficial cooling may be preferable used, but other embodiments are possible. A suitable range of wavelengths for the LOI treatment is the near-infrared range (900-3000 nm), with water serving as the main target chromophore.

(2) The LOI approach is thought to provide a significantly higher safety margin over the traditional approach between the threshold of therapeutic effect and the threshold of unwanted side effects. The safety margin is defined as Fₘₐₓ/Fₘᵢₙ, where Fₘᵢₙ is the threshold of the desired therapeutic effect and Fₘₐₓ is the threshold of the continuous bulk damage. The theoretical upper limit for the safety margin is 1/f, where f is the fill factor of the lattice. Practically, the safety margin is determined by the expression Fₘₐₓ=Fₘᵢₙ·(Tₘₐₓ−Tᵢ)/(Tₜᵣ−Tᵢ), where Tₘₐₓ is the temperature of water vaporization, Tₜᵣ is the minimal temperature, which still provides the therapeutic effect. This margin can be up to 2 times higher than in case of traditional photothermal treatment. It should also be emphasized that the periodicity of the lattice is important for keeping the safety margin stable and for maintaining reproducibility of results.

(3) The efficacy of the lattice treatment can be increased by minimizing the size of the islets and maximizing the fill factor of the lattice. Small-size spherical or elliptical islets can be produced by using wavelengths in the 900 to 1800 nm range and focusing technique with a high numerical aperture for depth in the tissue up to 0.7 mm with minimal irradiation of surface layers of the tissue. The positions of the optical islets correspond to the locations of ballistic foci. For deeper focusing, the ballistic focus disappears and the maximal irradiance stabilizes at ˜0.5 mm depth (the diffuse focus).

(4) Small size column-like islets can be created in the tissue using collimated micro beams. The confocal parameter of such a beam must be longer than the depth of column in the tissue. For depths exceeding 0.5 mm, the diameter of the micro beam is generally larger than 0.1 mm. In contrast with broad beams, the depth of penetration of the micro beams is relatively insensitive to the wavelength in the range 800-1800 nm. However, the threshold fluence for tissue damage depends strongly on the wavelength. The minimal threshold fluences can be found in the range between 1380 and 1570 nm. The depth of the resulting column can be controlled by the fluence. For a superficial column with 0.25 to 0.5 mm depth, the minimal threshold fluence can be achieved in the 1400-1420 nm wavelength range and the absolute value of this fluence is between 12 and 80 J/cm². For a deeper-penetrating column of a 0.75 mm depth, the minimal threshold fluences are found at 1405 nm (400 J/cm²) and 1530 nm (570 J/cm²). In principle, a LOI can be created at a depth up to several millimeters in tissue, but in this case the size of the islets will also grow to several millimeters.

(5) The extent of the optical damage is determined by the size of the optical islets and the fluence. A damage islet is collocated with the original optical islet if the pulse width is shorter than the thermal relaxation time of the optical islet and the fluence is close to the minimal effective fluence. For higher fluences, the damage islets can grow in size even after termination of the optical pulse and, as a result, the fill factors of LTI an LDI can be higher than the fill factor of the original LOI. Islets of a lattice can be created in tissue sequentially using scanner or concurrently using lattice of optical beams. In the latter case, the optimal pulse width is shorter than the thermal relaxation time of the lattice, approximately given by LTRT=TRT/3f where LTRT and TRT are the thermal relaxation times of the LOI and a single islet, respectively.

The concept of the lattices of optical islets can be used as a safe yet effective treatment modality in applications where the target of treatment is within the body and/or the location of irradiation of EMR is within the body. The same concept can be applied for other sources of energy such as microwave, radiofrequency, ultrasound, and others. Although the present embodiments are generally described with respect to electromagnetic radiation, it will be understood that embodiments using other forms of energy instead of or in addition to electromagnetic radiation are possible and are within the scope of the present invention.

### Lenses and Other Focusing Elements.

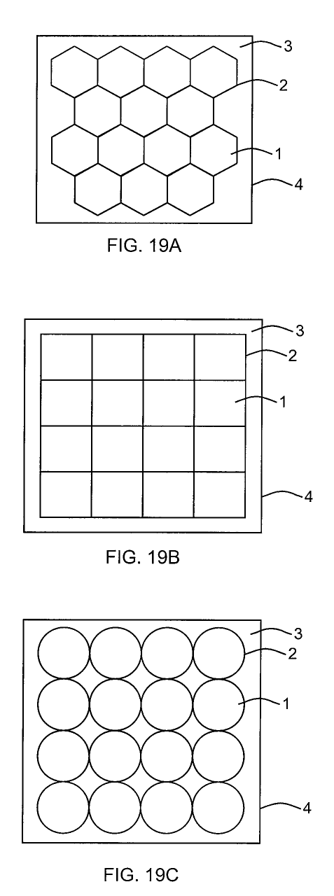

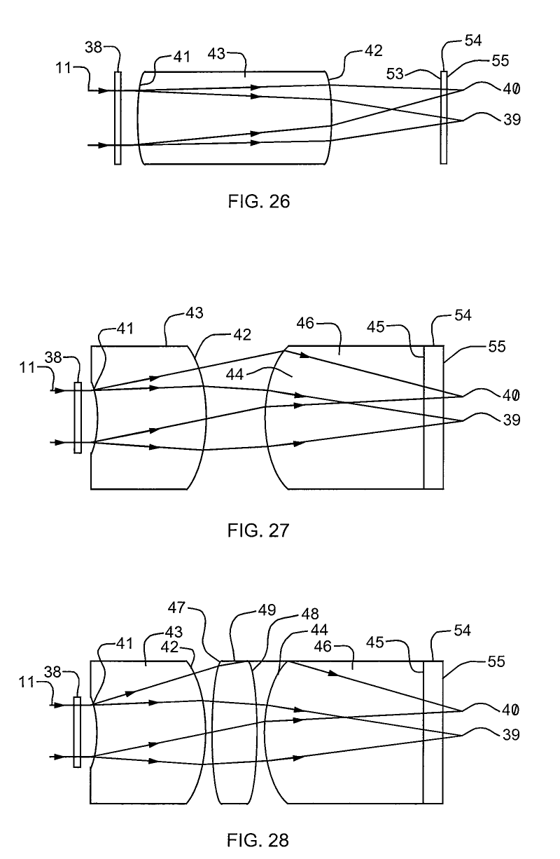

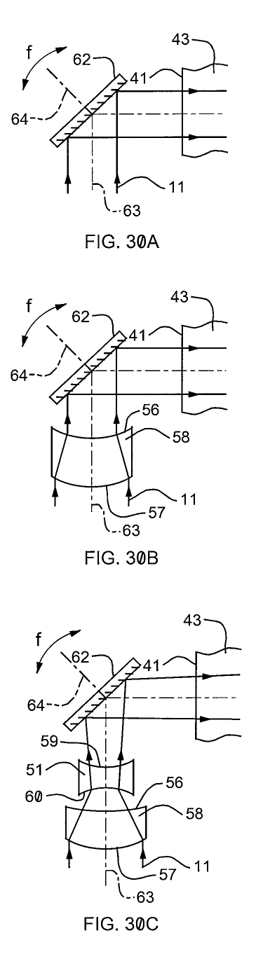

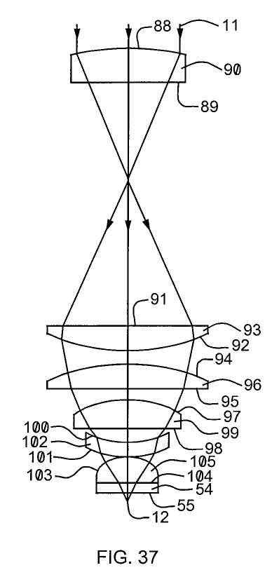

FIGS. 19A-25C illustrate various systems for delivering radiation in parallel to a plurality of target portions 214. The arrays of these figures are typically fixed focus arrays for a particular depth d. This depth may be changed either by using a different array having a different focus depth, by selectively changing the position of the array relative to the surface of the tissue or to target volume V or by controlling the amplitude-phase distribution of the incident radiation. FIGS. 26-29 show various optical lens arrays which may be used in conjunction with the scanning or deflector systems of FIGS. 30A-35 to move to successive one or more focused portions 214 within target volume V. Finally, FIGS. 36 and 37 show two different variable focus optical systems which may, for example, be moved mechanically or manually over the tissue to illuminate successive portions 214 thereon.

A. Focusing Elements

FIGS. 19A-C show a focusing element 1 on a substrate 3, the focusing element having a border which is in a hexagonal pattern (FIG. 19A), a square pattern (FIG. 19B), and a circular or elliptical pattern (FIG. 19C). Standard optical materials can be used for these elements. While the hexagonal and square patterns of FIGS. 19A and 19B can completely fill the working area of the focusing element plate 4, this is not true for the element pattern of FIG. 19C. Radiation from source 210 would typically be applied simultaneously to all of the focusing elements 1; however, the radiation may also be applied sequentially to these elements by use of a suitable scanning mechanism, or may be scanned in one direction, illuminating/irradiating for example four of the elements at a time.

B. Micro Lens Systems

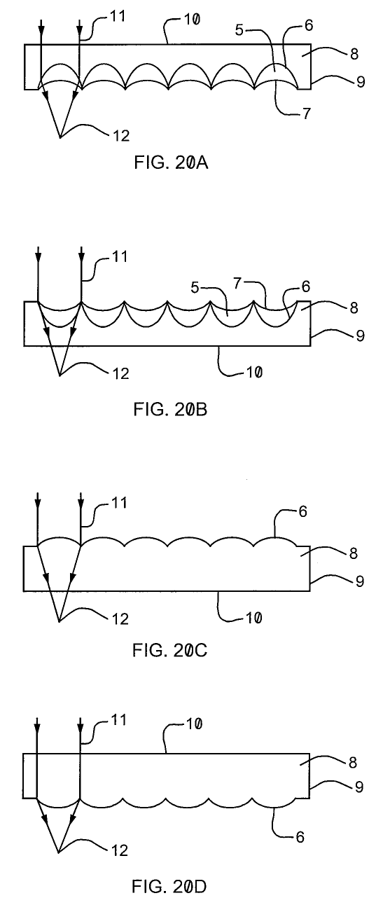

FIGS. 20A and 20B are cross-sectional views of a micro-lens system fused in a refracting material 8, for example, porous glass. The refractive index for the material of lenses 5 must be greater than the refractive index of refracting material 8. In FIG. 20B, beam 11 initially passes through planar surface 10 of refracting material 8 and is then refracted both by primary surface 6 and by secondary surface 7 of each micro-lens 5, resulting in the beam being focused to a focal point 12. The process is reversed in FIG. B20A, but the result is the same. In FIGS. 20C and 20D, the incident beam 11 is refracted by a primary lens surface 6 formed of the refracting material 8. Surfaces 6 and 7 for the various arrays can be either spherical or aspherical.

C. Lenses and Lens Arrays in Immersion Materials

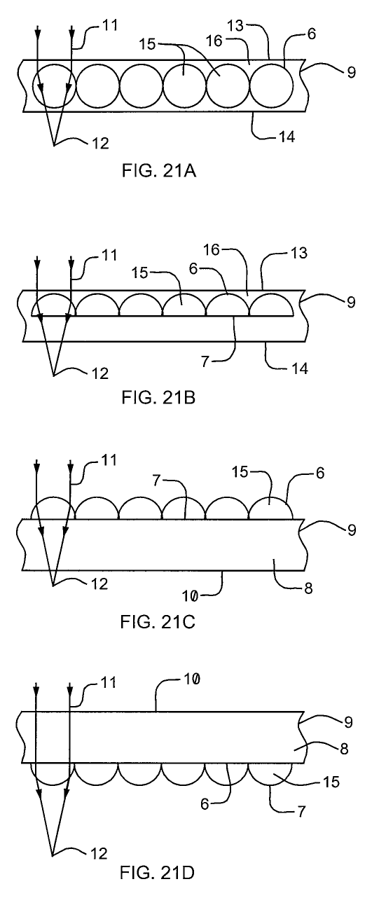

In FIGS. 21A and 21B, the lens pieces 15 are mounted to a substrate and are in an immersion material 16. The refraction index of lens pieces 15 are greater than the refraction index of immersion material 16. Immersion material 16 can be in a gas (air), liquid (water, cryogen spray) or a suitable solid gas and liquid can be used for cooling of the tissue. The immersion material is generally at the primary and secondary plane surfaces, 13 and 14, respectively. The focusing depth can be adjusted by changing the refractive index of immersion material. In FIG. 21B, the primary surface 6 and secondary surface 7 of each lens piece 15 allows higher quality focusing to be achieved. For FIGS. 21C and 21D, the lens pieces 15 are fixed on a surface of a refracting material 8, the embodiment of FIG. 21D providing a deeper focus than that of FIG. 21C, or that of any of other arrays shown in FIGS. 21B-21D for a given lens 15.

D. Fresnel Lenses

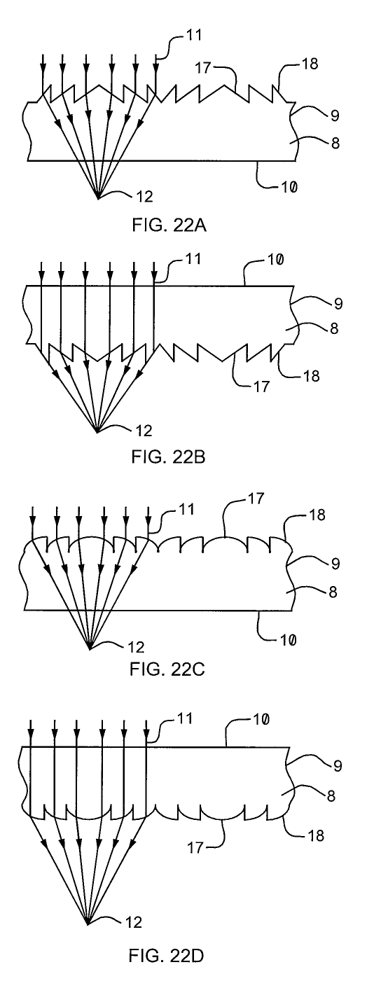

FIGS. 22A-D show Fresnel lens surfaces 17 and 18 formed on a refracting material 8. Changing the profile of Fresnel lens surface 17 and 18, the relationship between the radius of center 17 and ring 18 of the Fresnel surface, makes it possible to achieve a desired quality of focusing. The arrays of FIGS. 22C and 22D permit a higher quality focusing to be achieved and are other preferred arrays. Surfaces 17 and 18 can be either spherical or aspherical.

E. Holographic Lenses and Spatially Modulated Phase Arrays

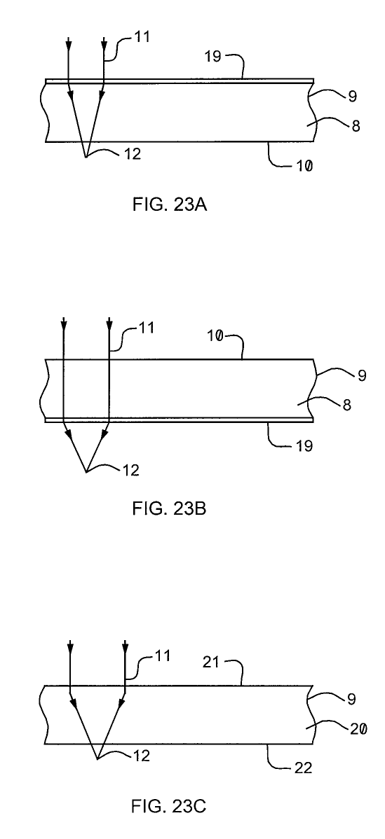

In FIGS. 23A and 23B, the focusing of an incident beam 11 is achieved by forming a holographic lens 19 on a surface of refracting material 8. Holographic lenses 19 may be formed on either of the surfaces of refracting material 8 as shown in FIGS. 23A and 23B or on both surfaces. FIG. 23C shows that the holographic material 20 substituted for the refracting material 8 of FIGS. 23A and 23B. The holographic lens is formed in the volume of material 20.

Techniques other than holography can be used to induce phase variations into different portions of the incident beam and, thus, provide amplitude modulation of the output beams.

F. Gradient Lenses

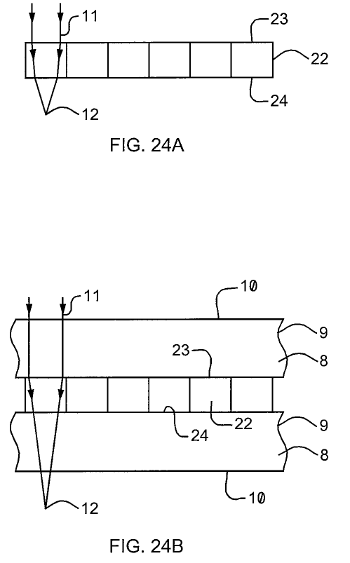

In FIGS. 24A and 24B, the focusing elements are formed by gradient lenses 22 having primary plane surfaces 23 and secondary plane surfaces 24. As shown in FIG. 24B, such gradient lenses may be sandwiched between a pair of refracting material plates 8 which provide support, protection and possibly cooling for the lenses.

G. Cylindrical Lenses

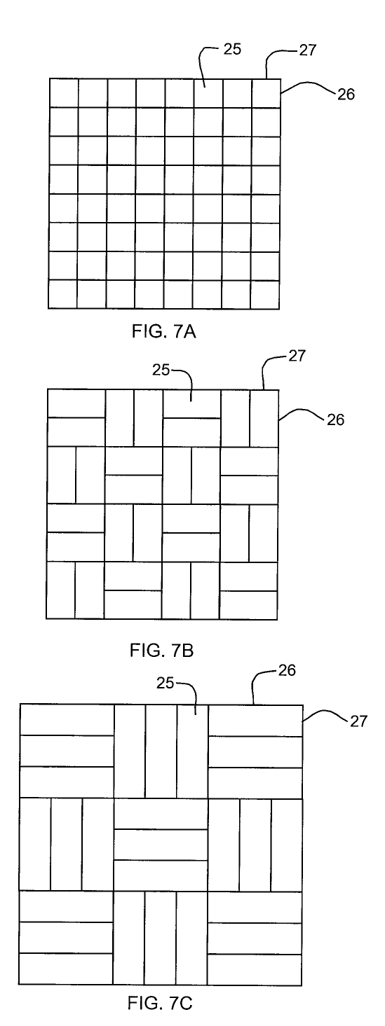

FIGS. 7A-7C illustrate various matrix arrays of cylindrical lenses 25. The relation of the lengths 26 and diameters 27 of the cylindrical lenses 25 can vary as shown in the figures. The cylindrical lens 25 of FIGS. 7B and 7C provide a line focus rather than a spot or circle focus as for the arrays previously shown.

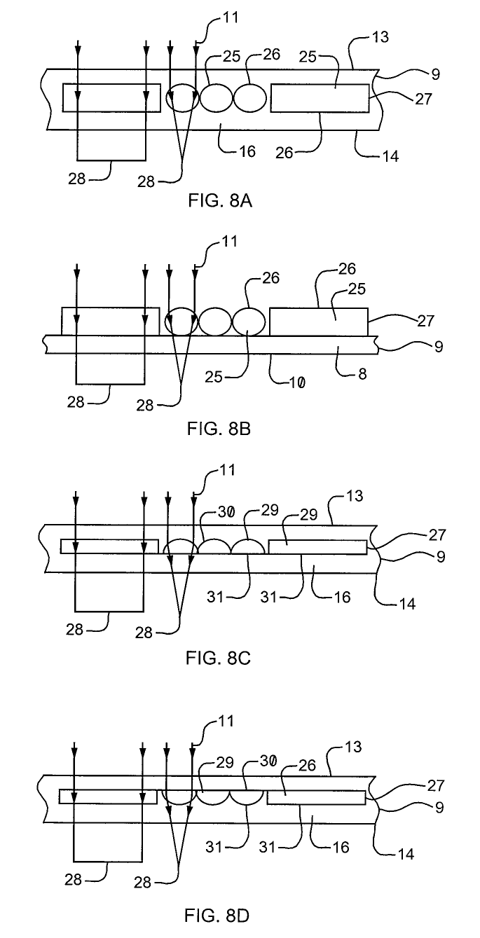

FIGS. 8A-8D are cross-sectional views of one layer of a matrix cylindrical lens system. The incident beam 11 is refracted by cylindrical lenses 25 (FIGS. 8A and 8B) or half cylinder lenses 29 (FIGS. 8C and 8D) and focus to a line focus 28. In FIGS. 8C and 8D, the cylindrical lenses 29 are in the immersion material 16. Primary working optical surface 30 and secondary optical working surface 31, which may be spherical or aspherical, allowing high quality focusing to be achieved. As shown in FIGS. 7A-8D the line focuses for adjacent lenses may be oriented in different directions, the orientations being at right angles to each other for certain of the lenses in these figures.

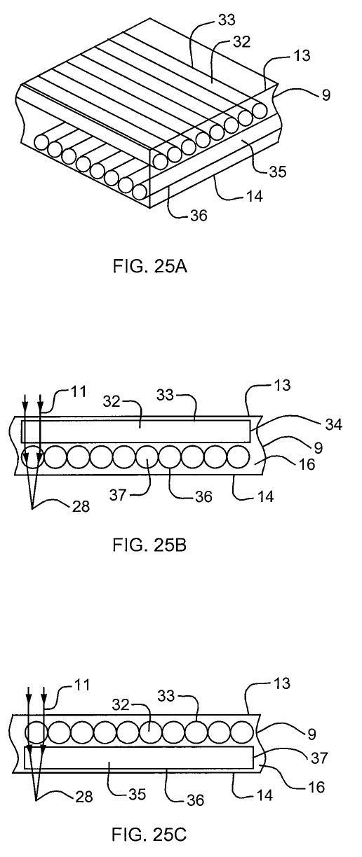

In FIGS. 25A, 25B and 25C, a matrix of focal spots is achieved by passing incident beam 11 through two layers of cylindrical lenses 32 and 35. FIGS. 25B and 25C are cross-sections looking in two orthogonal directions at the array shown in FIG. 25A. By changing the focal distance of primary layer lens 32, having a surface 33, and secondary lens 35, having a surface 36, it is possible to achieve a rectangular focal spot of a desired size. Primary layer lens 32 and secondary layer lens 35 are mounted in immersion material 16. Lenses 32 and 35 may be standard optical fibers or may be replaced by cylindrical lenses, which may be spherical or aspherical. Surfaces 34 and 37 can be of optical quality to minimize edge losses.

Described above optical system can be used with a pulse laser (0.1-100 ms) to introduce simultaneously into the tissue a lattice of optical islets. For example it can be an Er:glass laser (1.56 microns wavelength) or a Nd:YAG laser (1.44 microns) with fiber delivery and imaging optics to formed uniform beam before focusing elements.

H. One, Two, and Three-Lens Objectives

FIG. 26 shows a one-lens objective 43 with a beam splitter 38. The beam 11 incident on angle beam splitter (phase mask) 38 divides and then passes through the refracting surfaces 41 and 42 of lens 43 to focus at central point 39 and off-center point 40. Surfaces 41 and 42 can be spherical and/or aspherical. Plate 54 having optical planar surfaces 53 and 55 permits a fixed distance to be achieved between optical surface 55 and focusing points 39, 40. Angle beam splitter 38 can act as an optical grating that can split beam 11 into several beams and provide several focuses.

In FIG. 27, a two lens 43,46 objective provides higher quality focusing and numerical aperture as a result of optimal positioning of optical surfaces 41, 42 and 44. All of these surfaces can be spherical or aspherical. Optical surface 45 of lens 46 can be planar to increase numerical aperture and can be in contact with plate 54. Plate 54 can also be a cooling element as previously discussed.

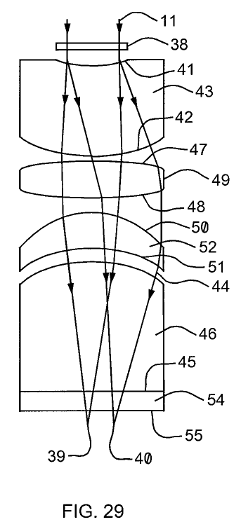

FIG. 28 differs from the previous figures in providing a three-lens objective, lenses 43, 46 and 49. FIG. 29 shows a four lens objective system, the optical surfaces 50 and 51 of lens 52 allowing an increased radius of treatment area (i.e., the distance between points 39 and 40).

I. Mirror-Containing Optical Systems

FIGS. 30A, 30B and 30C illustrate three optical systems, which may be utilized as scanning front ends to the various objectives shown in FIGS. 26-29. In these figures, the collimated initial beam 11 impinges on a scanning mirror 62 and is reflected by this mirror to surface 41 of the first lens 43 of the objective optics. Scanning mirror 62 is designed to move optical axis 63 over an angle f. Rotational displacement of a normal 64 of mirror 62 by an angle f causes the angle of beam 11 to be varied by an angle 2f. The optical position of scanning mirror 62 is in the entrance pupil of the focusing objective. To better correlate between the diameter of scanning mirror 62 and the radius of the working surface (i.e., the distance between points 39 and 40) and to increase the focusing quality, a lens 58 may be inserted before scanning mirror 62 as shown in FIG. 30B. Optical surfaces 56 and 57 of lens 58 can be spherical or aspherical. For additional aberration control, a lens 61 may be inserted between lens 58 and mirror 62, the lens 61 having optical surfaces 59 and 60.

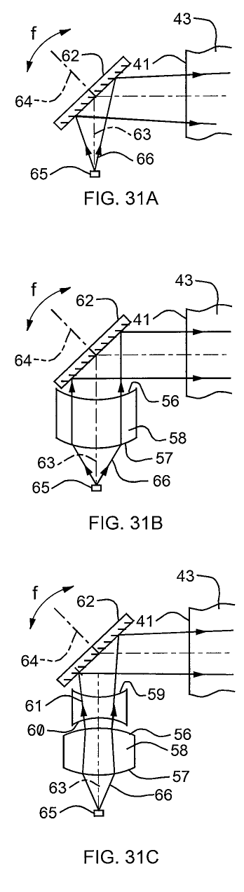

FIGS. 31A, 31B and 31C are similar to FIGS. 30A, 30B and 30C except that the light source is a point source or optical fiber 65 rather than collimated beam 11. Beam 66 from point source 65, for example the end of a fiber, is incident on scanning mirror 62 (FIG. 31A) or on surface 57 of lens 58 (FIGS. 31B and 31C).

J. Scanning Systems

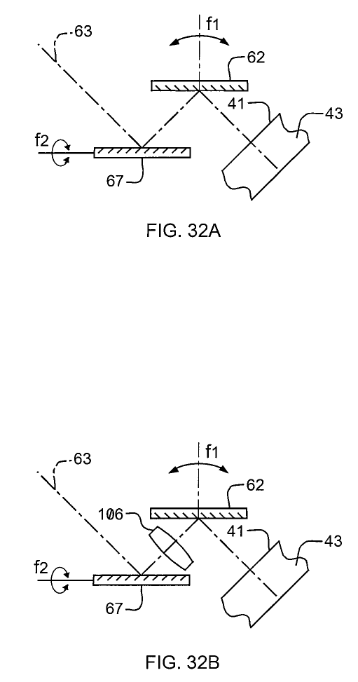

FIGS. 32A and 32B show a two mirror scanning system. In the simpler case shown in FIG. 32A, scanning mirror 67 rotates over an angle f2 and scanning mirror 62 rotates over an angle f1. Beam 63 is initially incident on mirror 67 and is reflected by mirror 67 to mirror 62, from which it is reflected to surface 41 of optical lens 43. In FIG. 32B, to increase the numerical aperture of the focusing beam, increase work area on the tissue and decrease aberration between scanning mirrors 62 and 67, an objective lens 106 is inserted between the mirrors. While a simple one-lens objective 106 is shown in this figure, more complex objectives may be employed. Objective lens 106 refracts the beam from the center of scanning mirror 67 to the center of scanning mirror 62.

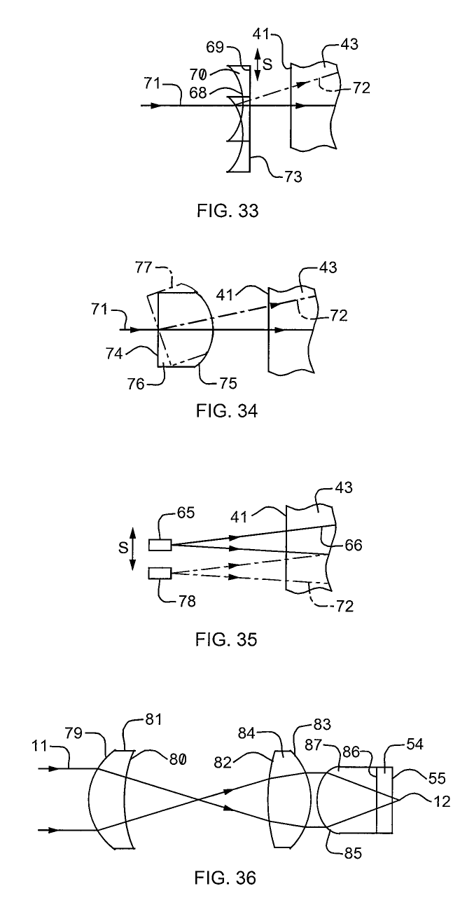

In FIG. 33, scanning is performed by scanning lens 70, which is movable in direction s. When scanning lens 70 is moved to an off center position 73, optical surface 68 refracts a ray of light along optical axis 71 to direction 72.

In FIG. 34, scanning is performed by rotating lens 76 to, for example, position 77. Surface 74 is planar and surface 75 is selected so that it does not influence the direction of refracted optical axis 72. In FIG. 35, scanning is performed by the moving of point source or optical fiber 65 in directions.

K. Zoom Lens Objectives

FIGS. 36 and 37 show zoom lens objectives to move the damage islets to different depths. In FIG. 36, a first component is made up of a single lens 81 movable along the optical axis relative to a second component, which is unmovable and consists of two lenses 84 and 87. Lens 84 is used to increase numerical aperture. To increase numerical aperture, range of back-focal distance and decrease focal spot size, optical surfaces 79, 80, 82, 83 and 85 can be aspherical. The relative position of the first and second components determines the depth of focal spot 12.

FIG. 37 shows zoom lens objectives with spherical optical surfaces. The first component is made up of a single lens 90 movable with respect to the second component along the optical axis. The second component, which is unmovable, consists of five lenses 93, 96, 99, 102, and 105. The radius of curvature of surfaces 88 and 89 are selected so as to compensate for aberrations of the unmovable second component. Again, the depth of focus may be controlled by controlling the distance between the first and second components. Either of the lens systems shown in FIGS. 36 and 37 may be mounted so as to be movable either manually or under control of control 218 to selectively focus on desired portions 214 of target volume V or to non-selectively focus on portions of the target volume.

L. Focus Depth.

While as may be seen from Table B1, depth d for volume V and the focal depth of an optical system are substantially the same when focusing to shallow depths, it is generally necessary in a scattering medium such as tissue to focus to a greater depth, sometimes a substantially greater depth, in order to achieve a focus at a deeper depth d. The reason for this is that scattering prevents a tight focus from being achieved and results in the minimum spot size, and thus maximum energy concentration, for the focused beam being at a depth substantially above that at which the beam is focused. The focus depth can be selected to achieve a minimum spot size at the desired depth d based on the known characteristics of the tissue.

M. Wavelength.

Both scattering and absorption are wavelength dependent. Therefore, while for shallow depths a fairly wide band of wavelengths can be utilized while still achieving a focused beam, the deeper the focus depth, the more scattering and absorption become factors, and the narrower the band of wavelengths available at which a reasonable focus can be achieved. Table B1 indicates preferred wavelength bands for various depths, although acceptable, but less than optimal, results may be possible outside these bands.

TABLE B1

| Depth of | | Numerical |
|---|---|---|
| damage, μm | Wavelength range, nm | Aperture range |
| 0-200 | 290-10000 | <3 |
| 200-300 | 400-1880 & 2050-2350 | <2 |
| 300-500 | 600-1850 & 2150-2260 | <2 |
| 500-1000 | 600-1370 & 1600-1820 | <1.5 |
| 1000-2000 | 670-1350 & 1650-1780 | <1 |
| 2000-5000 | 800-1300 | <1 |

N. Pulse Width.

Normally the pulse width of the applied radiation should be less than the thermal relaxation time (TRT) of each of the targeted portions or optical islets, since a longer duration may result in heat migrating beyond the boundaries of these portions. When relatively small islets are desired, pulse durations will also be relatively short. However, as depth increases, and the spot sizes thus also increase, maximum pulse width or duration also increase. The pulse-widths can be longer than the thermal relaxation time if density of the targets is not too high, so that the combined heat from the target areas at any point outside these areas is well below the damage threshold for tissue at such point. Generally, thermal diffusion theory indicates that pulse width τ for a spherical islet should be τ<500 D²/24 and the pulse width for a cylindrical islet with a diameter D is τ<50 D²/16, where D is the characteristic size of the target. Further, the pulse-widths can sometimes be longer than the thermal relaxation time if density of the targets is not too high, so that the combined heat from the target areas at any point outside these areas is well below the damage threshold for tissue at such point. Also, as will be discussed later, with a suitable cooling regimen, the above limitation may not apply, and pulse durations in excess of the thermal relaxation time, sometimes substantially in excess of TRT, may be utilized.

O. Power.

The required power from the radiation source depends on the desired therapeutic effect, increasing with increasing depth and cooling and with decreasing absorption due to wavelength. The power also decreases with increasing pulse width.

P. Numerical Aperture.

Numerical aperture is a function of the angle of a focused radiation beam from an optical device. (Not all embodiments require focusing, however.) It is preferable, but not essential, that this number, and thus the angle of the beam, be as large as possible so that the energy at portions in a tissue volume where radiation is concentrated is substantially greater than that at other points in the tissue volume V, thereby minimizing damage to the tissue in region being treated, and in portions of tissue volume V other than the EMR treated islets, while still achieving the desired therapeutic effect. Higher numerical aperture of the beam risk of damage to the integrity of the tissue and its function, but it is limited by scattering and absorption of higher incidence angle optical rays. As can be seen from Table B1, the preferable numerical aperture decreases as the focus depth increases.

### Additional Embodiments of Devices and Systems

In addition to the exemplary embodiments discussed earlier, many other embodiments are possible for internal treatments using EMR treated islets. Each device would be sized according to its intended purposes, and may be relatively large or, in some cases, small for performing treatments in certain parts of the body. A number of different devices and structures can be used to generate islets of treatment in the tissue.

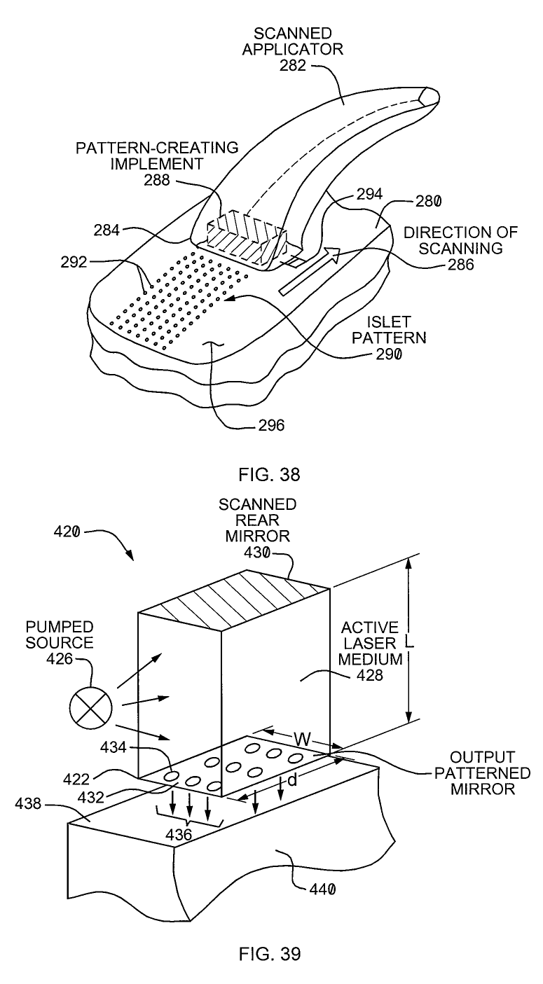

For example, FIG. 38 illustrates one system for producing the islets of treatment on tissue 280. An applicator 282 is provided with a handle so that its head 284 can be near or in contact with the tissue 280 and scanned in a direction 286 over the tissue 280. The applicator 282 can include an islet pattern generator 288 that produces a pattern of areas of enhanced permeability of the tissue or arrangement 290 of islets particles 292 on the surface of the tissue 280, which when treated with EMR from applicator 210 produces a pattern of enhanced permeability. In other embodiments, the generator 288 produces thermal, damage or photochemical islets into the surface or deeper layers or portions of the tissue.

In one embodiment, the applicator 282 includes a motion detector 294 that detects the scanning of the head 284 relative to the tissue surface 296. This generated information is used by the islet pattern generator 288 to ensure that the desired fill factor or islet density and power is produced on the tissue surface 296. For example, if the head 284 is scanned more quickly, the pattern generator responds by imprinting islets more quickly. The following description describes this embodiment, as well as other embodiments, in greater detail. Further, the following sections elaborate on the types of EMR sources that can be used with the applicator 282 and on the methods and structures that can be used to generate the islets of treatment.

Other embodiments may use one or more diode laser bars as the EMR source. Because many applications require a high-power light source, a standard 40-W, 1-cm-long, cw diode laser bar can be used in some embodiments. Any suitable diode laser bar can be used including, for example, 10-100 W diode laser bars. A number of types of diode lasers, such as those set forth above, can be used within the scope. Other sources (e.g., LEDs and diode lasers with SHG) can be substituted for the diode laser bar with suitable modifications to the optical and mechanical sub-systems.

FIG. 12A shows one embodiment using a diode laser bar. Many other embodiments can be used within the scope. In this embodiment, the hand piece 310 includes a housing 313, a diode laser bar 315, and a cooling or heating plate 317. The housing 313 supports the diode laser bar 315 and the cooling or heating plate 317, and the housing 313 can also support control features (not shown), such as a button to fire the diode laser bar 315. The housing 313 can be made from any suitable material, including, for example, plastics. The cooling plate, if used, can remove heat from the tissue. The heating plate, if used, can heat the tissue. The same plate can be used for heating or cooling, depending on whether a heat source or source of cooling is applied to the plate.

The diode laser bar 315 can be, in one embodiment, ten to fifty emitters (having widths of 50-to-150 μm in some embodiments or 100-to-150 μm in others) that are located along a 1-cm long diode bar with spacing of 50 to 900 μm. In other embodiments, greater than or less than fifty emitters can be located on the diode laser bar 315, the emitter spacing, and the length of the diode laser bar 315 can also vary. In addition, the width of the emitters can vary. The emitter spacing and the number of emitters can be customized during the manufacturing process.

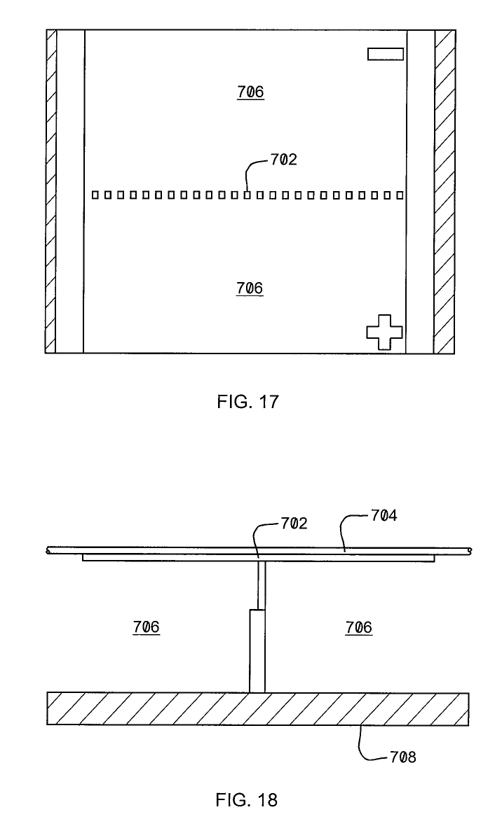

The diode laser bar 315 can be, in one embodiment, twenty-five 100-to-150 μm or 50-to-150 μm wide emitters that are located along a 1 cm long diode bar, each separated by around 50 to 900 microns in some embodiments, and approximately 500 microns in others. FIGS. 17 and 18 depict top and cross-sectional views, respectively, of such a diode laser bar assembly in this embodiment. In this embodiment, twenty-five emitters 702 are located directly beneath the surface plate 704 that is placed in contact with the tissue during treatment. Two electrodes 706 are located to each side of the emitters 702. The bottom of the diode assembly contains a cooling agent 708 to control the diode laser and plate 704 temperatures.

In the embodiment of FIGS. 17 and 18, the divergence of the beam emanating from the emitters 702 is between 6 and 12 degrees along one axis (the slow axis) and between 60 and 90 degrees along the fast axis. The plate 704 may serve as either a cooling or a heating surface and also serves to locate the emitters 702 in close and fixed proximity to the surface of the tissue to be treated. The distance between the emitters 702 and the plate 704 can be between about 50 and 1000 micrometers, and more particularly between about 100 and 1000 micrometers in some embodiments, in order to minimize or prevent distortion effects on the laser beam without using any optics for low cost and simplicity of manufacture. During use, the distance between the emitters 702 and the tissue can be between about 50 and 1000 micrometers, and more particularly 100 and 1000 micrometers in some embodiments. In such embodiments, imaging optics are not needed, but other embodiments may include additional optics to image the emitter surfaces 702 directly onto the tissue surface. In other embodiments, greater than or less than twenty-five emitters can be located on the diode laser bar, and the length of the diode laser bar can also vary. In addition, the width of the emitters and light divergence can vary. The emitter spacing and the number of emitters can be customized during the manufacturing process.

FIG. 12B shows a perspective view of one embodiment of a diode laser bar 330 that can be used for the diode laser bar 315 in FIG. 12A. The diode laser bar 330 has length L of around 1 cm, a width W of around 1 mm, and a thickness T of around 0.0015 mm. The depiction of FIG. 12B shows 12 emitters 332, each of which emits radiation 334 as shown in FIG. 12B. The diode laser bar 330 can be placed within the device 310 of FIG. 12A so that the side S of the diode laser bar 315 is oriented as shown in FIG. 12A. The emitters, therefore, emit radiation downward toward the tissue 319 in the embodiment of FIG. 12A.

Referring again to FIG. 12A, the plate 317 can be of any type, such as those set forth above, in which light from an EMR source can pass through the plate 317. In one embodiment, the plate 317 can be a thin sapphire plate. In other embodiments, other optical materials with good optical transparency and high thermal conductivity/diffusivity, such as, for example, diamond, can be used for the plate 317. The plate 317 can be used to separate the diode laser bar 315 from the tissue 319 during use. In addition, the plate 317 can provide cooling or heating to the tissue, if desired. The area in which the plate 317 touches the tissue can be referred to as the treatment window. The diode laser bar 315 can be disposed within the housing 313 such that the emitters are in close proximity to the plate 317, and therefore in close proximity to the tissue when in use.

In operation, one way to create islets of treatment is to place the housing 313, including the diode laser bar 315, in close proximity to the tissue, and then fire the laser. Wavelengths near 1750-2000 nm and in the 1400-1600 nm range can be used for creating subsurface islets of treatment with minimal effect on the epidermis due to high water absorption. Wavelengths in the 290-10,000 can be used in some embodiments, while in other wavelengths in the 900-10,000 nm range can be used for creating surface and subsurface islets on the tissue. Without moving the hand piece across the tissue, a series of treatment islets along a line can be formed in the tissue. FIG. 38 shows one possible arrangement 290 of islets on the surface of the tissue 280 from the use of such a diode laser bar, where the diode laser bar 315 is pulsed as it moves over the tissue in direction A of FIG. 12A.

In another embodiment, the user can simply place the hand piece in contact with the target tissue area and move the hand piece over the tissue while the diode laser is continuously fired to create a series of lines of treatment. For example, using the diode laser bar 330 of FIG. 12B, 12 lines of treatment would appear on the tissue (one line for each emitter).

In another embodiment, an optical fiber can couple to the output of each emitter of the diode laser bar. In such an embodiment, the diode laser bar need not be as close to the tissue during use. The optical fibers can, instead, couple the light from the emitters to the plate that will be in close proximity to the tissue when in use.

FIG. 12C shows another embodiment, which uses multiple diode laser bars to create a matrix of islets of treatment. As shown in FIG. 12C, multiple diode laser bars can be arranged to form a stack of bars 325. In FIG. 12C, for example, the stack of bars 325 includes five diode laser bars. In a similar manner as set forth above in connection with FIG. 12A, the stack of bars 325 can be mounted in the housing 313 of a hand piece H101 with the emitters very close to a cooling plate 317.

In operation, the hand piece 310 of FIG. 12C can be brought close to the tissue surface 319, such that the cooling plate 317 is in contact with the tissue. The user can simply move the hand piece over the tissue as the diode lasers are pulsed to create a matrix of islets of treatment in the tissue. The emission wavelengths of the stacked bars need not be identical. In some embodiments, it may be advantageous to mix different wavelength bars in the same stack to achieve the desired treatment results. By selecting bars that emit at different wavelengths, the depth of penetration can be varied, and therefore the islets of treatment spot depth can also be varied. Thus, the lines or spots of islets of treatment created by the individual bars can be located at different depths.

During operation, the user of the hand piece 310 of FIG. 12A or 12C can place the treatment window of the hand piece in contact with a first location on the tissue, fire the diode lasers in the first location, and then place the hand piece in contact with a second location on the tissue and repeat firing.

In addition to the embodiments set forth above in which the diode laser bar(s) is located close to the tissue surface to create islets of treatment, a variety of optical systems can be used to couple light from the diode laser bar to the tissue. For example, with reference to FIGS. 12A and 12C, imaging optics can be used to re-image the emitters onto the tissue surface, which allows space to be incorporated between the diode laser bar 315 (or the stack of bars 325) and the cooling plate 317. In another embodiment, a diffractive optic can be located between the diode laser bar 315 and the output window (i.e., the cooling plate 317) to create an arbitrary matrix of treatment spots. Numerous exemplary types of imaging optics and/or diffractive optics can also be used in this embodiment.

Another embodiment is depicted in FIG. 12D. In this embodiment, the housing 313 of the hand piece 310 includes a stack 325 of diode laser bars and a plate 317 as in previous embodiments. This embodiment, however, also includes four diffractive optical elements 330 disposed between the stack 250 and the plate 317. In other embodiments, more or fewer than four diffractive optical elements 330 can be included. The diffractive optical elements 330 can diffract and/or focus the energy from the stack 325 to form a pattern of islets of treatment in the tissue 319. In one aspect, one or more motors 334 is included in the hand piece 310 in order to move the diffractive optical elements 330. The motor 334 can be any suitable motor, including, for example, a linear motor or a piezoelectric motor. In one embodiment, the motor 334 can move one or more of the diffractive optical elements 330 in a horizontal direction so that those elements 330 are no longer in the optical path, leaving only one (or perhaps more) of the diffractive optical elements 334 in the optical path. In another embodiment, the motor 334 can move one or more of the diffractive optical elements 330 in a vertical direction in order to change the focusing of the beams.

In operation, by incorporating more than one diffractive optics 330 in the hand piece 310 along with a motor 334 for moving the different diffractive optics 330 between the stack 325 of diode laser bars and the plate 317, the diffractive optics 330 can be moved in position between the stack 325 and the cooling plate 317 in order to focus the energy into different patterns. Thus, in such an embodiment, the user is able to choose from a number of different islets of treatment patterns in the tissue through the use of the same hand piece 310. In order to use this embodiment, the user can manually place the hand piece 310 on the target area of the tissue prior to firing, similar to the embodiments described earlier. In other embodiments, the hand piece aperture need not tough the tissue. In such an embodiment, the hand piece may include a stand off mechanism (not shown) for establishing a predetermined distance between the hand piece aperture and the tissue surface.

FIG. 12E shows another embodiment. In this embodiment, optical fibers 340 are used to couple light to the output/aperture of the hand piece 310. Therefore, the diode laser bar (or diode laser bar stacks or other light source) can be located in a base unit or in the hand piece 310 itself. In either case, the optical fibers couple the light to the output/aperture of the hand piece 310.

In the embodiment of FIG. 12E, the optical fibers 340 may be bonded to the treatment window or cooling plate 317 in a matrix arrangement with arbitrary or regular spacing between each of the optical fibers 340. FIG. 12E depicts five such optical fibers 340, although fewer or, more likely, more optical fibers 340 can be used in other embodiments. For example, a matrix arrangement of 30 by 10 optical fibers may be used in one exemplary embodiment. In the depicted embodiment, the diode laser bar (or diode laser bar stacks) is located in the base unit (which is not shown). The diode laser bar (or diode laser bar stacks) can also be kept in the hand piece. The use of optical fibers 340 allow the bar(s) to be located at an arbitrary position within the hand piece 310 or, alternatively, outside the hand piece 310.

As an example of an application of a diode laser bar to create thermal damage zones in the epidermis of human tissue, a diode laser bar assembly, as depicted in FIGS. 17 and 18, emitting at a wavelength λ=1.47 μm, was constructed and applied to human tissue ex vivo at room temperature in a stamping mode (that is, in a mode where the assembly does not move across the tissue during use). The diode bar assembly had a sapphire window, which was placed in contact with the tissue and the laser was pulsed for about 10 ms. The treated tissue was then sliced through the center of the laser-treated zones to reveal a cross-section of the stratum corneum, epidermis and dermis. The resulting thermal damage channels were approximately 100 μm in diameter and 125-150 μm in depth for the 10 mJ per channel treatments.

In some embodiments using a Xe-filled linear flash lamp, the spectral range of the EMR is 300-3000 nm, the energy exposure up to 1000 J/cm², the pulse duration is from about 0.1 ms to 10 s, and the fill factor is about 1% to 90%.

Another embodiment involves the use of imaging optics to image the tissue and use that information to determine medication application rates, application of EMR, or the like in order to optimize performance. For instance, some medical treatments require that the medication application rate be accurately measured and its effect be analyzed in real time. The tissue surface imaging system can detect the size of reversible or irreversible holes created with techniques proposed in this specification for creating treatment islets in the stratum corneum. For this purpose, a capacitive imaging array can be used in combination with an image enhancing lotion and a specially optimized navigation/image processing algorithm to measure and control the application rate.

The use of a capacitive imaging array is set forth above in connection with FIG. 15. Such capacitive image arrays can be used, for example, within the applicator 282 of FIG. 38 according to this embodiment. As set forth above, in addition to measuring hand piece speed, the capacitive imaging arrays 350, 352 (FIG. 15) can also image the tissue. Acquired images can be viewed in real time during treatment via a display window of the device.

One example of a suitable capacitive sensor for this embodiment is a sensor having an array of 8 image-sensing rows by 212 image-sensing columns. A typical capacitive array sensor is capable of processing about 2000 images per second. To allow for processing images in real time, an orientation of the sensor can be selected to aid in functionality. In one embodiment, for instance, the images are acquired and processed along the columns.

FIGS. 39 and 40 illustrate still other exemplary embodiments in which the islets of treatment are formed generally through the use of a mirror containing holes or other transmissive portions. Light passes through the holes in the mirror and strikes the tissue, creating islets of treatment. Light reflected by the mirror stays in the optical system and through a system of reflectors is re-reflected back toward the mirror which again allows light to pass through the holes. In this manner, the use of a mirror containing holes can be more efficient than the use of a mask with holes, where the mask absorbs rather than reflects light.

In the embodiment of FIG. 39, the patterned optical radiation to form the islets of treatment is generated by a specially designed laser system 420 and an output mirror 422. The laser system 420 and output mirror 422 can be contained in, for instance, a hand piece. In other embodiments, the laser system 420 can be contained in a base unit and the light passing through the holes in the mirror can be transported to the hand piece aperture through a coherent fiber optic cable. In still other embodiments, the laser can be mounted in the hand piece and microbeams from the laser can be directed to the tissue with an optical system. In the illustrated embodiment, the laser system 420 comprises a pump source 426, which optically or electrically pumps the gain medium 428 or active laser medium. The gain medium 428 is in a laser cavity defined by rear mirror 430 and output mirror 422. Any type of EMR source, including coherent and non-coherent sources, can be used in this embodiment instead of the particular laser system 420 shown in FIG. 39.

According to one aspect, the output mirror 422 includes highly reflective portions 432 that provide feedback (or reflection) into the laser cavity. The output mirror 422 also includes highly transmissive portions 434, which function to produce multiple beams of light that irradiate the surface 438 of the tissue 440. FIG. 39 depicts the highly transmissive portions 434 as being circular shapes, although other shapes, including, for example, rectangles, lines, or ovals, can also be used. The transmissive portions 434 can, in some embodiments, be holes in the mirror. In other examples, the transmissive portions 434 include partially transparent micro mirrors, uncoated openings, or openings in the mirror 422 with an antireflection coating. In these embodiments, the rest of the output mirror 422 is a solid mirror or an uncoated surface.

In one implementation, the output mirror 422 functions as a diffractive multi-spot sieve mirror. Such an output mirror 422 can also serve as a terminal or contact component of the optical system 420 to the surface 438 of the tissue 440. In other embodiments, the output mirror 422 can be made from any reflective material.

Because of the higher refractive index of the illuminated tissue of the tissue 440, divergence of the beams is reduced when it is coupled into the tissue 440. In other embodiments, one or more optical elements (not shown) can be added to the mirror 422 in order to image a sieve pattern of the output mirror 422 onto the surface of the tissue 440. In this latter example, the output mirror 422 is usually held away from the tissue surface 438 by a distance dictated by the imaging optical elements.

Proper choice of the laser cavity length L, operational wavelength λ of the source 426, the gain g of the laser media 428, dimensions or diameter D of the transmissive portions 434 (i.e., if circular) in the output mirror 422, and the output coupler (if used) can help to produce output beams 436 with optimal properties for creating islets of treatment. For example, when D2/4λL<1, effective output beam diameter is made considerably smaller than D, achieving a size close to the system's wavelength λ of operation. This regime can be used to produce any type of treatment islets.

Typically, the operational wavelength ranges from about 0.29 μm to 100 μm and the incident fluence is in the range from 1 mJ/cm² to 100 J/cm². The effective heating pulse width can be in the range of less than 100 times the thermal relaxation time of a patterned compound (e.g., from 100 fsec to 1 sec).

In other embodiments, the chromophore layer is not used. Instead the wavelength of light is selected to directly create the pathways.

In one example, the spectrum of the light is in the range of or around the absorption peaks for water. These include, for example, 970 nm, 1200 nm, 1470 nm, 1900 nm, 2940 nm, and/or any wavelength >1800 nm. In other examples, the spectrum is tuned close to the absorption peaks for lipids, such as 0.92 μm, 1.2 μm, 1.7 μm, and/or 2.3 μm, and wavelengths like 3.4 μm, and longer or absorption peaks for proteins, such as keratin, or other endogenous tissue chromophores contained in the tissue.

The wavelength can also be selected from the range in which this absorption coefficient is higher than 1 cm⁻¹, such as higher than about 10 cm⁻¹. Typically, the wavelength ranges from about 0.29 μm to 100 μm and the incident fluence is in the range from 1 mJ/cm² to 1000 J/cm². The effective heating pulse width is preferably less than 100× thermal relaxation time of the targeted chromophores (e.g., from 100 fsec to 1 sec).

FIGS. 10A-10C show another embodiment in which the output EMR from the hand piece is totally internally reflected when the hand piece is not in contact with a tissue. When the hand piece is in contact with a tissue, the output EMR is spatially modulated in order to create islets of treatment in the tissue.

The embodiment of FIGS. 10A-10C can include an EMR source 542, an optical reflector 546, one or more optical filters 548, a light duct 550 (or concentrator), and a cooling plate (not pictured). The total internal reflection in the embodiment of FIGS. 10A-10C is caused by the shape of the distal end 544 of the light duct 550. The distal end 550 can be an array of prisms, pyramids, hemispheres, cones, etc. . . . , such as set forth in FIGS. 10B and 10C. The array of elements have dimensions and shapes that introduce light total internal reflection (TIR) when the distal end 544 is in a contact with air, as shown in FIG. 10B. In contrast, the distal end 544 does not cause TIR (it frustrates TIR) when the distal end 544 is in a contact with a lotion or tissue surface, as shown in FIG. 10C. Further, when the distal end 544 is in a contact with a lotion or tissue surface, this leads to light spatial modulation and concentration of the EMR in a contact area of the tissue, causing islets of treatment.

In the exemplary embodiment of FIG. 10A-10C using a Xe-filled linear flash lamps, the spectral range of electromagnetic radiation is about 300-3000 nm, the energy exposure is up to about 1000 J/cm², the laser pulse duration is from about 0.1 ms to 10 seconds, and the fill factor is from about 1% to 90%.

The embodiments of FIGS. 10A, 10B, and 10C depict the use of a non-coherent light source in a hand piece. However, a mechanism can also be used to cause TIR in an embodiment using a coherent light source, such as, for example, a solid state laser or a diode laser bar. Referring to the embodiments of FIGS. 12A-E, 15 and 16, the light from the diode laser bar 315 (in FIG. 12A) can also be coupled to the tissue via a total internal reflection (TIR) prism. Since the diode laser bar 315 might not be located in close proximity to the tissue surface, an optical system might be required to re-image the emitters onto the tissue. Thus, a distal end with prisms or the like can be used to re-image the emitters onto the tissue. In one embodiment, a TIR prism can be used. When the TIR prism is not in contact with tissue, light from the diode laser bar would be internally reflected and no light would be emitted from the hand piece. However, when the tissue is coated with an index-matching lotion and the tissue is brought into contact with the hand piece (and, in particular, the prism), light is coupled into the tissue. Thus, in a manner similar to that described above for non-coherent light sources, TIR reflection prisms or arrays can also be used in embodiments using coherent light sources. This feature can be important for eye and tissue safety.

G. Solid State Laser Embodiments

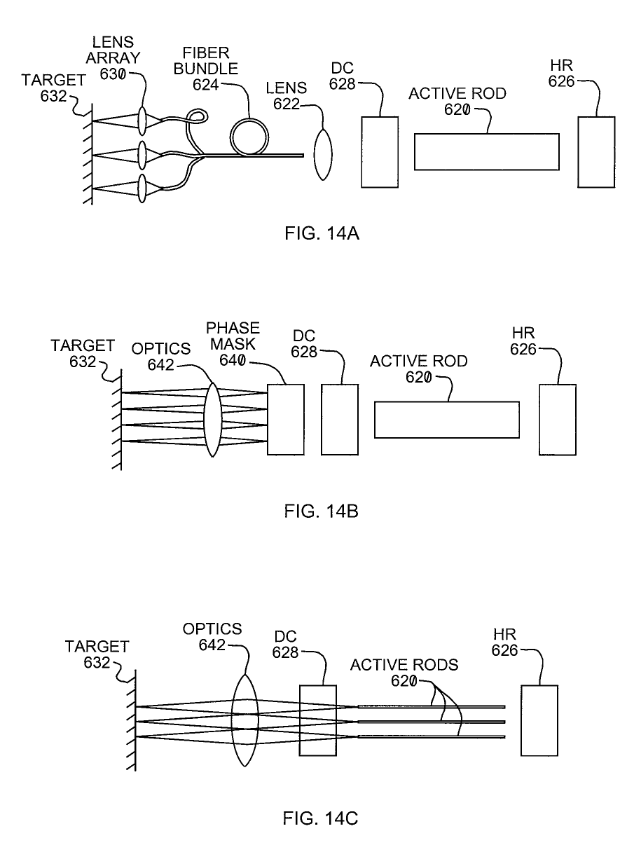

FIGS. 14A, 14B, and 14C show additional embodiments. FIG. 14A shows an embodiment in which the apparatus includes a laser source 620, focusing optics (e.g., a lens) 622, and a fiber bundle 624. The laser source 620 can be any suitable source for this application, for example, a solid state laser, a fiber laser, a diode laser, or a dye laser. In one embodiment, the laser source 620 can be an active rod made from garnet doped with rare earth ions. The laser source 620 can be housed in a hand piece or in a separate base unit.

In the exemplary embodiment as in FIG. 14A, the laser source 620 is surrounded by a reflector 626 (which can be a high reflector HR) and an output coupler 628 (OC). In other embodiments, the reflector 626 and the coupler 628 are not used. Various types and geometries of reflectors can be used for reflector 626. The fiber bundle 624 is located optically downstream from the lens 622, so that the optical lens 622 directs and focuses light into the fiber bundle 624.

In one embodiment, an optical element 630, such as a lens array, can be used to direct and output the EMR from the fiber bundle 624 in order to focus the EMR onto the tissue 632. The optical element 630 can be any suitable element or an array of elements (such as lenses or micro lenses) for focusing EMR. In the embodiment of FIG. 14A, the optical element 630 is a micro lens array. In other embodiments, an optical element 630 might not be used. In such an embodiment, the outputs of the fibers in the fiber bundle 624 can be connected to one side of a treatment window (such as a cooling plate of the apparatus), where the other side of the treatment window is in contact with the tissue 632.

In operation, the laser source 620 generates EMR and the reflector 626 reflects some of it back toward the output coupler 628. The EMR then passes through the output coupler 628 to the optical lens 622, which directs and focuses the EMR into the fiber bundle 624. The micro lens array 630 at the end of the fiber bundle 624 focuses the EMR onto the tissue 632.

FIG. 14B shows another embodiment. In this embodiment, the apparatus includes a laser source 620 and a phase mask 640. The laser source 620 can be any type of laser source and can be housed in a hand piece or in a separate base unit, such as in the embodiment of FIG. 14A. In one embodiment, the laser source 620 can be an active rod made from garnet doped with rare earth ions. Also like the embodiment of FIG. 14A, the laser source 620 can be surrounded by a reflector 626 (which can be a high reflector HR) and can output EMR into an output coupler 628 (OC).

The embodiment of FIG. 14B includes a phase mask 640 that is located between the output coupler 628 and an optical element 642. The phase mask 640 can include a set of apertures that spatially modulate the EMR. Various types of phase masks can be used in order to spatially modulate the EMR in order to form islets of treatment on the tissue 632. The optical element 642 can be any suitable element or an array of elements (such as lenses or micro lenses) that focuses the EMR radiation onto the tissue 632. In embodiment of FIG. 14B, the optical element 642 is a lens.

In operation, the laser source 620 generates EMR and the reflector 626 reflects some of it back toward the output coupler 628. The EMR then passes through the output coupler 628 to the phase mask 640, which spatially modulates the radiation. The optical element 642, which is optically downstream from the phase mask 640 so that it receives output EMR from the phase mask 640, generates an image of the apertures on the tissue.

FIG. 14C shows another embodiment. In this embodiment, the apparatus includes multiple laser sources 650 and optics to focus the EMR onto the tissue 632. The multiple laser sources 650 can be any suitable sources for this application, for example, diode lasers or fiber lasers. For example, the laser sources 650 can be a bundle of active rods made from garnet doped with rare earth ions. The laser sources 650 can optionally be surrounded by a reflector and/or an output coupler, similar to the embodiments of FIGS. 14A and 14B.

In the embodiment of FIG. 14C, an optical element 642 can be used for focusing the EMR onto the tissue 632. Any suitable element or an array of elements (such as lenses or micro lenses) can be used for the optical element 642. The optical element, for example, can be a lens 642.

In operation, the bundle of lasers 650 generate EMR. The EMR is spatially modulated by spacing apart the laser sources 650 as shown in FIG. 14C. The EMR that is output from the laser sources 650, therefore, is spatially modulated. This EMR passes through the output coupler 628 to the optical element 642, which focuses the EMR onto the tissue 632 to form islets of treatment.

In the exemplary embodiment of FIGS. 14A, 14B, and 14C, which each use a garnet laser rod doped with rare earth ions, the spectral range of electromagnetic radiation is about 400-3000 nm, the energy exposure is up to about 1000 J/cm², the laser pulse duration is from about 10 ps to 10 s, and the fill factor is from about 1% to 90%.

Several sets of exemplary parameters for treatment according to some embodiments of the invention are provided in Table D1.

TABLE D1
Exemplary Treatment Parameters

| Damage | | | | |
|---|---|---|---|---|
| heating depth, | | | | |
| mm | 1 | 2 | 3 | 5 |
| Damage/heated | 0.2-3 | 0.5-5 | 0.75-6 | 1-10 |
| zone diameter, | | | | |
| mm | | | | |
| Wavelength, | 900-1850 | 900-1400 | 900-1350 | 900-1300 |
| nm | 2080-2300 | 1500-1750 | | |
| Beam diameter | 0.5-8 | 1-10 | 2-15 | 3-25 |
| (2D beam) or | | | | |
| width (1D | | | | |
| beam), mm | | | | |
| Fill factor* | 0.01-0.5 | 0.01-0.3 | 0.01-0.3 | 0.01-0.3 |
| Pulse width, s | 0.001-10 | 0.1-20 | 0.5-30 | 1-120 |
| Precooling | 0-10 | 0-20 | 0-60 | 0-100 |
| time, s | | | | |
| Postcooling | 0-20 | 0-30 | 0-60 | 0-120 |
| time, s | | | | |
| Input power | 5-100 | 3-70 | 1-50 | 0.5-35 |
| density, W/cm² | | | | |

In some embodiments using a flash lamp, the technical specifications can be as summarized in Table E1 below. These embodiments can be used for a number of applications.

TABLE E1
Exemplary Parameters for Flashlamps

| Specification | Symbol | Value | Units |
|---|---|---|---|
| Incident Fluence | Finc | 1-25 | J/cm² |
| Wavelength Range (of EMR | λₘᵢₙ, λₘₐₓ | 400-2000 | nm |
| source) | | | |
| Spot Size (of optical absorbers) | SS | 1-50 dia. | mm |
| Pulse width (of EMR source) | PW | 1-1000 | ms |
| Lifetime | Tlife | 10-10000 | pulses |
| Number of Lamps (of EMR | #lamps | 1-10 | # |
| source) | | | |
| Pulse Period (of EMR source) | T | 1-10 | sec |
| Island/mesh Diameter | ID | 10-100 | um |
| Pattern pitch | PP | 100-5000 | um |

### EQUIVALENTS

While only certain embodiments have been described, it will be understood by those skilled in the art that various changes in form and details may be made therein without departing from the spirit and scope as defined by the appended claims. Those skilled in the art will recognize, or be able to ascertain using no more than routine experimentation, many equivalents to the specific embodiments described specifically herein. Such equivalents are intended to be encompassed in the scope of the appended claims.

### REFERENCES AND DEFINITIONS

The patent, scientific and medical publications referred to herein establish knowledge that was available to those of ordinary skill in the art at the time the invention was made. The entire disclosures of the issued U.S. patents, published and pending patent applications, and other references cited herein are hereby incorporated by reference in their entirety.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent or later-developed techniques which would be apparent to one of skill in the art. In addition, in order to more clearly and concisely describe the claimed subject matter, the following definitions are provided for certain terms which are used in the specification and appended claims.

Numerical Ranges. As used herein, the recitation of a numerical range for a variable is intended to convey that the embodiments may be practiced using any of the values within that range, including the bounds of the range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values ≧0 and ≦2 if the variable is inherently continuous. Finally, the variable can take multiple values in the range, including any sub-range of values within the cited range.

Or. As used herein, unless specifically indicated otherwise, the word “or” is used in the inclusive sense of “and/or” and not the exclusive sense of “either/or.”

As used herein, EMR includes the range of wavelengths approximately between 200 nm and 10 mm. Optical radiation, i.e., EMR in the spectrum having wavelengths in the range between approximately 200 nm and 100 μm, is preferably employed in the embodiments described above, but, also as discussed above, many other wavelengths of energy can be used alone or in combination. The term “narrow-band” refers to the electromagnetic radiation spectrum, having a single peak or multiple peaks with FWHM (full width at half maximum) of each peak typically not exceeding 10% of the central wavelength of the respective peak. The actual spectrum may also include broad-band components, either providing additional treatment benefits or having no effect on treatment. Additionally, the term optical (when used in a term other than term “optical radiation”) applies to the entire EMR spectrum. For example, as used herein, the term “optical path” is a path suitable for EMR radiation other than “optical radiation.”

It should be noted, however, that other energy may be used to for treatment islets in similar fashion. For example, non EMR sources such as ultrasound, photo-acoustic and other sources of energy may also be used to form treatment islets. Thus, although the embodiments described herein are described with regard to the use of EMR to form the islets, other forms of energy to form the islets are within the scope of the invention and the claims.

What is claimed is:

1. A method of treating internal tissue comprising:
accessing an internal tissue volume to be treated; and
irradiating portions of the internal tissue volume with electromagnetic radiation;
wherein the electromagnetic radiation causes the heated portions to form islets of treated tissue surrounded by untreated tissue.

2. The method of claim 1, wherein the internal tissue is accessed by one of an incision, an open wound, and an orifice of a body cavity.

3. The method of claim 1, wherein the internal tissue is a tissue from the group muscle, cartilage, ligaments, bone, fat, dermis, blood vessels, nervous tissue, gastrointestinal, heart, lungs, kidney, gall bladder, and liver.

4. The method of claim 1, wherein the heated portions are ablated.

5. The method of claim 1, wherein the heated portions are coagulated.

6. The method of claim 1, wherein the heated portions are denatured.

7. The method of claim 1, wherein the heated portions are heated without further damage to the tissue in the heated portions.

8. The method of claim 1, wherein the treated tissue if welded.

9. The method of claim 1, wherein the treated tissue is surgical incision.

10. The method of claim 1, wherein the treated tissue is composed of two portions of tissue joined during surgery.

11. The method of claim 1, wherein the heated portions are heated substantially simultaneously.

12. The method of claim 1, wherein the portions are irradiated for a duration that is greater than the thermal relaxation time of the tissue volume to be treated.

13. A method of treating internal tissue comprising:
inserting a treatment device into the internal tissue to be treated; and
causing the treatment device to transmit electromagnetic radiation from the device to portions of the internal tissue;
forming subvolumes of damaged tissue corresponding to the irradiated portions of the internal tissue, wherein the subvolumes are separated by undamaged tissue.

14. The method of claim 13, wherein the treatment device is a device from the group of cannulas and catheters.

15. The method of claim 13 wherein the internal tissue to be treated is a blood vessel, and the treatment device is inserted into a lumen of the blood vessel.

16. The method of claim 13 wherein the subvolumes are separated such that the ratio of the subvolumes to the volume of internal tissue being treated is between about 0.1% and about 90%.

17. The method of claim 13 wherein the subvolumes are separated such that the ratio of the subvolumes to the volume of internal tissue being treated is between about 10% to about 50%.

18. The method of claim 13 wherein the subvolumes are separated such that the ratio of the subvolumes to the volume of internal tissue being treated is between about 10% to about 30%.

19. A method for performing a treatment on a volume located at area and depth coordinates of an internal tissue of a patient including:
providing a source of treatment radiation; and
applying treatment radiation from said source to an optical system providing multiple foci for concentrating said radiation to at least one depth within said depth coordinate and to selected areas within said area coordinates of said volume such that following application of the treatment radiation three dimensionally located treatment portions are formed at said foci in said volume separated from one another by untreated portions of said volume.

20. A method for performing a treatment on a volume located at area and depth coordinates of an internal tissue by irradiating portions of the volume including:
providing a source of treatment radiation;
precooling the internal tissue over at least part of the area coordinate to a selected temperature for a selected duration, the selected temperature and duration being sufficient to cool the internal tissue to a depth below the depth coordinate to a temperature below normal body temperature of the internal tissue; and
applying the treatment radiation to an optical system having a plurality of foci which concentrates said radiation to at least one depth coordinate and to selected areas within said area coordinate to define treatment portions at said foci in said volume following application of the treatment radiation, said treatment portions being less than said volume, each said treatment portion being within untreated portions and being substantially surrounded by cooled internal tissue separating said treatment portion from other treatment portions.

21. A device for performing a treatment on a volume of internal tissue located at area and depth coordinates of a patient's skin including:
a source of treatment radiation; and
an optical system to which treatment radiation from said source is applied, said optical system providing a plurality of foci for concentrating said treatment radiation to at least one depth in said volume of internal tissue and to selected areas of said volume, said at least one depth and said areas defining three dimensional treatment portions at said foci in said volume within untreated portions of said volume,
a controller for selectively activating said source so as to successively irradiate said plurality of foci.

22. The device of claim 21, further comprising a cooling system configured to cool the volume of internal tissue.

23. The device of claim 22, wherein the cooling system is configured to cool the volume of internal tissue during operation to a selected temperature and to a selected depth.

24. The device of claim 21 wherein the device further comprises a cannula configured to emit radiation from a portion thereof.

25. The device of claim 21, wherein the device further comprises a catheter configured to emit radiation from a portion thereof.
